(19) United States
(12) Patent Application Publication
(10) Pub. No.: US 2022/0240518 A1
(43) Pub. Date: Aug. 4, 2022

(54) ALGICIDAL SHEWANELLA BACTERIA OR ITS FILTRATE IMMOBILIZED TO POROUS MATRICES AND USES THEREOF

(71) Applicants: Kathryn J. Coyne, Milford, DE (US); Yanfei Wang, Lewes, DE (US)

(72) Inventors: Kathryn J. Coyne, Milford, DE (US); Yanfei Wang, Lewes, DE (US)

(73) Assignee: University of Delaware, Newark, DE (US)

(21) Appl. No.: 17/619,356

(22) PCT Filed: Jun. 26, 2020
(86) PCT No.: PCT/US2020/039953

§ 371 Date: Dec. 15, 2021

Related U.S. Application Data
(60) Provisional application No. 62/867,251, filed on Jun. 27, 2019.
(60) Provisional application No. 62/970,289, filed on Feb. 5, 2020.

(51) Int. Cl. A01N 63/20; C02F 3/34; C12N 1/20; C12N 11/10

(52) U.S. Cl. CPC A01N 63/20; C02F 3/348; C02F 1/50; C12N 11/10; C12N 1/20

(57) ABSTRACT

The present invention provides an algicidal composition comprising Shewanella strain IRI-160 or a filtrate of a Shewanella strain IRI-160 culture, a matrix and a medium. The Shewanella strain IRI-160 or the filtrate is immobilized to the matrix. Also provided are methods for preparing the algicidal composition and using the algicidal composition for inhibiting growth of a dinoflagellate.

### CROSS-REFERENCE TO RELATED APPLICATION

This application claims priority to U.S. Provisional Application No. 62/867,251, filed Jun. 27, 2019, and U.S. Provisional Application No. 62/970,289, filed Feb. 5, 2020, and the contents of each of which are incorporated herein by reference in their entireties for all purposes.

### REFERENCE TO U.S. GOVERNMENT SUPPORT

This invention was made with government support under Grant Nos. NA180AR4170086, NA15NOS4780176 and NA10NOS4780136 from the National Oceanic and Atmospheric Administration. The United States has certain rights in the invention.

### FIELD OF THE INVENTION

The invention relates to algicidal compositions comprising Shewanella bacteria or a filtrate thereof and preparation and uses thereof for preventing and controlling harmful dinoflagellate blooms.

### BACKGROUND OF THE INVENTION

Harmful algal blooms (HABs) pose a threat to marine organisms and human health worldwide and are continuing to expand globally. Multiple approaches have been developed to prevent, control and mitigate HABs, including nutrient manipulation, clay flocculation, sonication, and application of toxic chemicals such as copper sulfate. Despite the effectiveness of these methods, they can be costly or raise concerns about negative effects on other organisms in the environment. To address these issues, biological approaches have been developed and applied to control HABs, including methods involving algicidal bacteria.

The algicidal bacterium, Shewanella sp. IRI-160, isolated from Delaware's inland bays has been shown to control the growth of dinoflagellates, for example, Pfiesteria piscicida, Prorocentrum minimum, and Lavenderina fissa (aka Gyrodinium instriatum), while having no negative effects on the growth of other algal taxa tested, including diatom, raphidophyte, prasinophyte, and cryptophyte species (Hare et al., in Harmful Algae 4:221-34 (2005)). This bacterium secretes water-soluble algicidal compounds, designated as IRI-160AA, and the filtrate of Shewanella sp. IRI-160 culture has been demonstrated to control the growth of dinoflagellates without the requirement of direct bacteria-algae contact (Pokrzywinski et al., in Harmful Algae 19:23-29 (2012)). IRI-160AA exhibits a significantly greater inhibitory effect on dinoflagellates K. veneficum and L. fissa in the exponential phase compared to the stationary phase, suggesting the potential application of this algicide for prevention and control of harmful dinoflagellate blooms during early phases of bloom development. IRI-160AA has negative impacts on nuclear and chromosome structures in dinoflagellates, as well as on chloroplasts, PSII and photosynthetic transport chain of photosynthetic dinoflagellates. Cell death was also accompanied by DNA degradation, reactive oxygen species production, cell cycle arrest, and DEVD-ase activity, suggesting a programmed pathway leading to cell death. Ammonium and several amines were identified in the algicide, each of which may play a role individually or together with other compounds in the algicide to contribute to the algicidal activity. Recently, IRI-160AA was tested on organisms at higher trophic levels, including copepods, fish, and shellfish. No negative impacts were observed at concentrations required to control the growth of dinoflagellates. This research provided further support for the application of this bacterium and its algicide as an environmentally neutral means to control HABs.

Although the algicidal activity and mechanisms of cell death for dinoflagellates exposed to Shewanella sp. IRI-160 or IRI-160AA as well as their effects on non-target species have been extensively investigated, there remains a need for an environmentally neutral approach for application of Shewanella sp. IRI-160 or IRI-160AA to effectively control harmful dinoflagellates in a natural environment without biosafety concerns.

### SUMMARY OF THE INVENTION

The present invention relates to algicidal compositions comprising Shewanella strain IRI-160 or a filtrate of a Shewanella strain IRI-160 culture, and preparation and uses thereof.

A first algicidal composition for inhibiting growth of a dinoflagellate in an environment is provided. The first algicidal comprises an effective amount of Shewanella strain IRI-160, a matrix and a medium. The Shewanella strain IRI-160 is immobilized to the matrix.

The first algicidal composition may comprise the Shewanella strain IRI-160 in an amount effective for maintaining a cell abundance of the dinoflagellate in the environment for at least 6 days at no more than 80% of a cell abundance of the dinoflagellate in the environment treated with a control composition, and the control composition may comprise the matrix and the medium but not the Shewanella strain IRI-160. The first algicidal composition may comprise the Shewanella strain IRI-160 at a concentration of at least 10⁸ cells per mL. The first algicidal composition may retain at least 80% of the Shewanella strain IRI-160 after storage of the first algicidal composition at a temperature of 4 ° C. for at least 14 days.

In the first algicidal composition, the matrix may comprise an agent selected from the group consisting of alginate, agarose, cellulose, polyester and a combination thereof. The first algicidal composition may be in a form selected from the group consisting of alginate beads, agarose cubes, cellulosic sponge, polyester foam and combinations thereof.

A second algicidal composition for inhibiting growth of a dinoflagellate in an environment is provided. The second algicidal composition comprises a filtrate of a Shewanella strain IRI-160 culture, a matrix and a medium. The filtrate is immobilized to the matrix.

The second algicidal composition may comprise the filtrate in an amount effective for maintaining a cell abundance of the dinoflagellate in the environment for at least 6 days at no more than 80% of a cell abundance of the dinoflagellate in the environment treated with a control composition, and the control composition may comprise the matrix and the medium but not the filtrate. The Shewanella strain IRI-160 culture may comprise Shewanella strain IRI-160 at a concentration of at least 10⁸ cells per mL. The second algicidal composition may retain at least 80% of the filtrate after storage of the algicidal composition at a temperature of 4 ° C. for at least 14 days.

In the second algicidal composition, the matrix may comprise an agent selected from the group consisting of alginate, agarose and a combination thereof. The second algicidal composition may be in a form selected from the group consisting of alginate beads, agarose cubes and a combination thereof. In one embodiment, the matrix comprises alginate. In another embodiment, the first or second algicidal composition is in a form of alginate beads.

The first or second algicidal composition may be effective for inhibiting growth of the dinoflagellate in the environment after storage of the algicidal composition at a temperature of 4° C. for at least 14 days.

In the first or second algicidal composition, the dinoflagellate may be selected from the group consisting of genus Karlodinium, Gyrodiunium, Pfiesteria, Alexandrium, Cochlodinium, Dinophysis, Karenia, Prorocentrum, Heterocapsa, and Oxyrrhis. The dinoflagellate may be selected from the group consisting of species Pfiesteria piscicida, Karlodinium veneficum, Alexandrium fundyense, Alexandrium tamarense, Cochlodinium polykrikoides, Cochlodinium polykrikoides, Dinophysis acuminate, Gyrodinium instriatum, Gyrodinium uncatenum, Heterocapsa triquetra, Karenia brevis, Lavenderina fissa (aka Gyrodinium instriatum and Gyrodinium uncatenum), Oxyrrhis marina and Prorocentrum minimum.

In the first or second algicidal composition, the medium may be a casein amino acid (CAA) medium at a concentration between 1× and 10×. The medium may comprise natural seawater, f/2 nutrients and casein amino acids.

A first preparation method for preparing the first algicidal composition is provided. The first preparation method comprises immobilizing the Shewanella strain IRI-160 in the medium to the matrix. As a result, the first algicidal composition is prepared.

A second preparation method for preparing the second algicidal composition is provided. The second preparation method comprises immobilizing the filtrate of the Shewanella strain IRI-160 culture in the medium to the matrix. As a result, the second algicidal composition is prepared.

In an algicidal composition prepared according to the first or second preparation method, the matrix may comprise an agent selected from the group consisting of alginate, agarose and a combination thereof. The algicidal composition may be in a form selected from the group consisting of alginate beads, agarose cubes and a combination thereof. In one embodiment, the matrix comprises alginate. In another embodiment, the algicidal composition is in a form of alginate beads.

The algicidal composition prepared according to the first or second preparation method may be effective for inhibiting growth of the dinoflagellate in the environment after storage of the algicidal composition at a temperature of 4° C. for at least 14 days.

According to the first or second preparation method, the dinoflagellate may be selected from the group consisting of genus Karlodinium, Gyrodiunium, Pfiesteria, Alexandrium, Cochlodinium, Dinophysis, Karenia, Prorocentrum, Heterocapsa, and Oxyrrhis. The dinoflagellate may be selected from the group consisting of species Pfiesteria piscicida, Karlodinium veneficum, Alexandrium fundyense, Alexandrium tamarense, Cochlodinium polykrikoides, Cochlodinium polykrikoides, Dinophysis acuminate, Gyrodinium instriatum, Gyrodinium uncatenum, Heterocapsa triquetra, Karenia brevis, Lavenderina fissa (aka Gyrodinium instriatum and Gyrodinium uncatenum), Oxyrrhis marina and Prorocentrum minimum.

According to the first or second preparation method, the medium may be a CAA medium at a concentration between 1× and 10×. The medium may comprise natural seawater, f/2 nutrients and casein amino acids.

A first treatment method for inhibiting growth of a dinoflagellate in an environment is provided. The first treatment method comprises applying an effective amount of an algicidal composition to the dinoflagellate in the environment. The algicidal composition comprises Shewanella strain IRI-160, a matrix and a medium, and the Shewanella strain IRI-160 is immobilized to the matrix.

The first treatment method may further comprise maintaining a cell abundance of the dinoflagellate in the environment at no more than 80% of a cell abundance of the dinoflagellate in the environment treated with a control composition for at least 6 days, and the control composition may comprise the matrix and the medium but not the Shewanella strain IRI-160.

A second treatment method for inhibiting growth of a dinoflagellate in an environment is provided. The second treatment method comprises applying an effective amount of an algicidal composition to the dinoflagellate in the environment. The algicidal composition comprises a filtrate of a Shewanella strain IRI-160 culture, a matrix and a medium, and the filtrate is immobilized to the matrix.

The second treatment method may further comprise maintaining a cell abundance of the dinoflagellate in the environment at no more than 80% of a cell abundance of the dinoflagellate in the environment treated with a control composition for at least 6 days, and the control composition may comprise the matrix and the medium but not the filtrate.

The first or second treatment method may further comprise storing the algicidal composition at a temperature of 4° C. for at least 14 days before applying the algicidal composition.

According to the first or second treatment method, the matrix may comprise an agent selected from the group consisting of alginate, agarose and a combination thereof. The algicidal composition is in a form selected from the group consisting of alginate beads, agarose cubes and a combination thereof. In one embodiment, the matrix may comprise alginate. In another embodiment, the algicidal composition may be in a form of alginate beads.

According to the first or second treatment method, the dinoflagellate may be selected from the group consisting of genus Karlodinium, Gyrodiunium, Pfiesteria, Alexandrium, Cochlodinium, Dinophysis, Karenia, Prorocentrum, Heterocapsa, and Oxyrrhis. The dinoflagellate may be selected from the group consisting of species Pfiesteria piscicida, Karlodinium veneficum, Alexandrium fundyense, Alexandrium tamarense, Cochlodinium polykrikoides, Cochlodinium polykrikoides, Dinophysis acuminate, Gyrodinium instriatum, Gyrodinium uncatenum, Heterocapsa triquetra, Karenia brevis, Lavenderina fissa (aka Gyrodinium instriatum and Gyrodinium uncatenum), Oxyrrhis marina and Prorocentrum minimum.

According to the first or second treatment method, the medium may be a CAA medium at a concentration between 1× and 10×. The medium may comprise natural seawater, f/2 nutrients and casein amino acids.

### BRIEF DESCRIPTION OF THE DRAWINGS

FIG. 1 shows the cell density of Shewanella sp. IRI-160 immobilized in alginate beads (A) or agarose cubes (B), or onto sponge (C) or polyester cubes (D), compared to free-living bacteria that were not immobilized (E) at 4 (left) and 25° C. (right) over 12 days. Results for the initial (Day 0; D0) and the last day (Day 12; D12) of experiments are shown. Error bars indicate standard deviations of three replicates, except that only two replicates were used in the D12 data of Shewanella sp. IRI-160 immobilized to agarose cubes (B) due to the contamination of one sample. Asterisks “*” indicate significant differences between cell densities of Shewanella sp. IRI-160 immobilized to each matrix (A-D) or the free-living cells (E) on D0 and D12 (p<0.05).

FIG. 2 shows specific growth rates of treatments (harmful dinoflagellates Karlodinium veneficum and Prorocentrum minimum, as well as non-harmful control cryptophyte Rhodomonas sp. treated with free-living or immobilized Shewanella sp. IRI-160 [10⁶ to 10⁸ cells mL⁻¹]) and controls (treated with blank alginate beads with no bacteria) over 6 days. Asterisks “*” indicate significant differences between specific growth rates of treatments and controls (p<0.05).

FIG. 3 shows Shewanella sp. IRI-160 density (cells per bead) in alginate beads in algal cultures on Day 0 (D0) and Day 6 (D6) for the 10⁸ cells mL⁻¹ immobilized bacteria treatments. Insert: Bacterial density (cells per bead) on D6 in blank alginate beads added to non-axenic control cultures. Error bars indicate standard deviations of three replicates. Asterisks “*” indicate significant differences (p<0.05) between bacterial cell abundance per bead on D0 and D6 (grey bars), or between bacterial cell abundance per bead in control cultures (insert) on D6 (white bars) compared to D0 (not shown).

FIG. 4 shows total bacterial cell densities in the medium of each treatment relative to non-axenic control cultures (dashed line) on Day 6 for dinoflagellates Karlodinium veneficum and Prorocentrum minimum, and cryptophyte Rhodomonas sp. Samples were incubated with Shewanella sp. IRI-160 immobilized in alginate beads (10⁶ to 10⁸ cells mL⁻¹) or free-living Shewanella sp. IRI-160 at 10⁸ cells mL⁻¹. Control algal cultures received blank beads only (with no bacteria). Error bars indicate standard deviations of three replicates. Asterisks “*” indicate significant differences in relative cell densities of total bacteria in the medium compared to controls on Day 6 (p<0.05).

FIG. 5 shows ammonium concentrations in each treatment relative to controls (black dashed line) in cultures of dinoflagellates Karlodinium veneficum and Prorocentrum minimum, as well as cryptophyte Rhodomonas sp., on Day 6. Samples were incubated with free-living Shewanella sp. IRI-160 (10⁸ cells mL⁻¹) or Shewanella sp. IRI-160 immobilized in alginate beads (10⁶ to 10⁸ cells mL⁻¹), compared to the control algal cultures incubated with blank beads only. Error bars indicate standard deviations of three replicates. Asterisk “*” indicates a significant difference between relative ammonium concentration in the indicated group and control cultures (p<0.05).

FIG. 6 shows in vivo fluorescence measurements of harmful dinoflagellates Karlodinium veneficum (A) and Prorocentrum minimum (B), as well as non-harmful control species Rhodomonas sp. (C) when incubated with Shewanella sp. IRI-160 (10⁶ to 10⁸ cells mL⁻¹) immobilized in alginate beads, compared to blank beads (control) and free-living Shewanella sp. IRI-160 at 10⁸ cells mL⁻¹. Error bars indicate standard deviations of three replicates. Asterisks “*” indicate significant differences between in vivo fluorescence of treatments vs. control at indicated time points (p<0.05).

FIG. 7 shows ammonium concentrations in treatments and controls in cultures of dinoflagellates Karlodinium veneficum and Prorocentrum minimum, as well as cryptophyte Rhodomonas sp., on Day 6. Samples were incubated with free-living Shewanella sp. IRI-160 (10⁸ cells mL⁻¹) or Shewanella sp. IRI-160 immobilized in alginate beads (10⁶ to 10⁸ cells mL⁻¹), compared to the control algal cultures incubated with blank beads only. Error bars indicate standard deviations of three replicates. Asterisk “*” indicates a significant difference between ammonium concentrations in the indicated group and control cultures (p<0.05).

FIG. 8 shows in vivo fluorescence of harmful dinoflagellate Karlodinium veneficum and non-harmful control cryptophyte species Rhodomonas sp. when incubated over 3 days with alginate beads prepared with cell-free algicide IRI-160AA compared to blank beads (no algicide control). Error bars indicate standard deviations (N=3). Asterisks indicate significant differences (p<0.05).

### DETAILED DESCRIPTION OF THE INVENTION

The present invention relates to algicidal compositions in which Shewanella sp. IRI-160 or a filtrate of a Shewanella sp. IRI-160 culture is immobilized to a matrix. The invention was made based on the inventors' surprising discovery of long lasting algicidal compositions by mixing alginate solution in CAA medium with algicidal Shewanella sp. IRI-160 or directly dissolving alginic acid with the algicidal filtrate of Shewanella sp. IRI-160 in 10X concentrated CAA medium such that the algicidal Shewanella sp. IRI-160 or its algicidal filtrate is immobilized to a matrix, which improves retention of Shewanella sp. IRI-160 or its filtrate over time while preserving selective algicidal effects on harmful dinoflagellate blooms. The inventors have investigated the retention of Shewanella sp. IRI-160 within several porous matrices (e.g., agarose, alginate hydrogel, cellulosic sponge and polyester foam) stored at different temperatures. The effects of the immobilized bacteria to alginate hydrogel at different densities were tested on cultures of various dinoflagellates and were compared with the algicidal activity of the free-living algicidal bacteria. The inventors have also investigated the algicidal effects of the Shewanella sp. IRI-160 filtrate immobilized to alginate hydrogel on dinoflagellates. The resulting algicidal compositions may be used to control or inhibit growth of dinoflagellates and prevent the harmful algal blooms caused by these species.

The Shewanella strain IRI-160 is a bacterial isolate from the Delaware Inland Bays. Shewanella strain IRI-160 is widespread in the coastal environment, and most likely found at high salinity, associated with particles. Shewanella strain IRI-160 shows increased abundance when dinoflagellates are present. The preparation and maintenance of the Shewanella strain IRI-160 have been described previously by Hare et al. in Harmful Algae 4:221-34 (2005), the content of which is incorporated herein by reference in its entirety. The 16S rRNA gene sequence of the Shewanella strain IRI-160 is available at GenBank Accession No. AY566557.

For example, to isolate this species, seawater may be filtered onto a 3-micron pore-size filter, and the bacteria may be transferred onto an LM plate. The bacteria can be individually sequenced or all of the bacteria on the plate can be interrogated with a labeled probe to identify those having the 16S rDNA of the Shewanella strain IRI-160.

A Shewanella strain IRI-160 culture may be obtained using conventional techniques. For example, a colony of the Shewanella strain IRI-160 may be transferred to any suitable medium (e.g., LM medium) and incubated under suitable conditions (e.g., at 20-24° C.) until, for example, a mid to late exponential phase.

In one embodiment, Shewanella sp. IRI-160 is grown in LM medium to mid-log growth stage. The mixture is then centrifuged to remove the medium. The cell pellet is then washed twice with a seawater medium (amended with nutrients), then resuspended in the seawater medium and incubated at 25° C. for one week. The cell culture was then filtered through a 0.2 pm membrane filter to remove the cells to generate an algicidal filtrate of the Shewanella strain IRI-160 culture. The filtrate of the Shewanella strain IRI-160 culture may be stored at −80° C. until use.

The term “dinoflagellate” used herein refers to any dinoflagellate species, for example, a harmful dinoflagellate species. Examples of dinoflagellates include those in genus Karlodinium, Gyrodiunium, Pfiesteria, Alexandrium, Cochlodinium, Dinophysis, Karenia, Prorocentrum, Heterocapsa, and Oxyrrhis. Other examples include the dinoflagellate species Pfiesteria piscicida, Karlodinium veneficum, Alexandrium fundyense, Alexandrium tamarense, Cochlodinium polykrikoides, Cochlodinium polykrikoides, Dinophysis acuminate, Gyrodinium instriatum, Gyrodinium uncatenum, Heterocapsa triquetra, Karenia brevis, Lavenderina fissa (aka Gyrodinium instriatum and Gyrodinium uncatenum), Oxyrrhis marina and Prorocentrum minimum.

The term “inhibiting growth of a dinoflagellate in an environment” as used herein means reducing a cell abundance or growth rate of the dinoflagellate in the environment. The environment may be an aquatic system, for example, a body of fresh water or a marine environment.

The term “immobilizing” or “immobilized” used herein refers to attachment of Shewanella strain IRI-160 to a matrix (e.g., agarose, alginate, sponge or polyester) such that the bacterium may be associated with a matrix, for example, on a surface of a matrix (e.g., sponge or polyester) or embedded in a matrix (e.g., alginate or agarose). The term “immobilizing” or “immobilized” used herein also refers to the capturing of the filtrate of Shewanella strain IRI-160 to a matrix (e.g., agarose and alginate) such that the filtrate may be embedded in the matrix. For example, Shewanella strain IRI-160 may be associated with a matrix on its surface or embedded in a matrix while a filtrate of a Shewanella strain IRI-160 culture may be embedded in a matrix.

The present invention provides a first algicidal composition for inhibiting growth of a dinoflagellate in an environment. The first algicidal composition comprises Shewanella strain IRI-160, a matrix and a medium. The Shewanella strain IRI-160 is immobilized to the matrix. The Shewanella strain IRI-160 is present in an amount effective for inhibiting growth of the dinoflagellate in the environment.

The first algicidal composition may comprise the Shewanella strain IRI-160 in an amount effective for reducing the cell abundance or growth rate of the dinoflagellate in the environment as compared with a control composition. The control composition may be the same as the first algicidal composition except that the Shewanella strain IRI-160 is not immobilized to the matrix in the control composition.

In the first algicidal composition, the Shewanella strain IRI-160 may be present in an amount effective for maintaining a cell abundance of the dinoflagellate in the environment at no more than 50%, 60%, 70%, 80%, 90% or 95% of a cell abundance of the dinoflagellate in the environment treated with a control composition over a period of, for example, at least 1, 2, 3, 4, 5, 6, 7, 8, 9, 10, 11, 12, 13, 14, 15, 16, 17, 18, 19, 20, 21 or 28 days, or at least 1, 2, 3, 4, 5, 6, 7 or 8 weeks. In one embodiment, the first algicidal composition comprises the Shewanella strain IRI-160 in an amount effective for maintaining a cell abundance of the dinoflagellate in the environment for at least 6 days at no more than 80% of a cell abundance of the dinoflagellate in the environment treated with a control composition, and the control composition comprises the matrix and the medium but not the Shewanella strain IRI-160.

In the first algicidal composition, the Shewanella strain IRI-160 may be present in an amount effective for maintaining a growth rate of the dinoflagellate in the environment at no more than 50%, 60%, 70%, 80%, 90% or 95% of a growth rate of the dinoflagellate in the environment treated with a control composition over a period of, for example, at least 1, 2, 3, 4, 5, 6, 7, 8, 9, 10, 11, 12, 13, 14, 15, 16, 17, 18, 19, 20, 21 or 28 days, or at least 1, 2, 3, 4, 5, 6, 7 or 8 weeks. In one embodiment, the first algicidal composition comprises the Shewanella strain IRI-160 in an amount effective for maintaining a growth of the dinoflagellate in the environment for at least 6 days at no more than 80% of a growth rate of the dinoflagellate in the environment treated with a control composition, and the control composition comprises the matrix and the medium but not the Shewanella strain IRI-160.

The first algicidal composition may comprise the Shewanella strain IRI-160 at a concentration of at least 10¹⁰, 10⁹, 10⁸, 10⁷, 10⁶, 10⁵ or 10⁴ cells per mL. In one embodiment, the first algicidal composition may comprise the Shewanella strain IRI-160 at a concentration of at least 10⁸ cells per mL.

In the first algicidal composition, the Shewanella strain IRI-160 is algicidal. The first algicidal composition may retain a desirable percentage of the algicidal Shewanella strain IRI-160 after storage. The algicidal composition may be stored at a temperature from 4° C. to 30° C. for a period of, for example, at least 1, 2, 3, 4, 5, 6, 7, 8, 9, 10, 11, 12, 13, 14, 15, 16, 17, 18, 19, 20, 21 or 28 days, or at least 1, 2, 3, 4, 5, 6, 7 or 8 weeks. At least 50%, 55%, 60%, 65%, 70%, 75%, 80%, 85%, 90% or 95% of the algicidal Shewanella strain IRI-160 may be retained after the storage. In one embodiment, the first algicidal composition retains at least 80% of the algicidal Shewanella strain IRI-160 after storage at 4° C. for at least 14 days.

The first algicidal composition may remain effective for inhibiting growth of a dinoflagellate in an environment after storage. The first algicidal composition may be stored at a temperature from 4° C. to 30° C. for a period of, for example, at least 1, 2, 3, 4, 5, 6, 7, 8, 9, 10, 11, 12, 13, 14, 15, 16, 17, 18, 19, 20, 21 or 28 days, or at least 1, 2, 3, 4, 5, 6, 7 or 8 weeks. The first algicidal composition may remain at least 50%, 55%, 60%, 65%, 70%, 75%, 80%, 85%, 90% or 95% effective for inhibiting growth of a dinoflagellate in an environment after storage. In one embodiment, the first algicidal composition remain effective for inhibiting growth of a dinoflagellate in an environment after storage at 4° C. for at least 14 days.

The present invention also provides a second algicidal composition for inhibiting growth of a dinoflagellate in an environment. The second algicidal composition comprises a filtrate of a Shewanella strain IRI-160 culture, a matrix and a medium. The filtrate is immobilized to the matrix. The filtrate is present in an amount effective for inhibiting growth of the dinoflagellate in the environment.

The second algicidal composition may comprise the filtrate in an amount effective for reducing a cell abundance or growth rate of the dinoflagellate in the environment as compared with a control composition. The control composition may be the same as the second algicidal composition except that the filtrate is not immobilized to the matrix in the control composition.

In the second algicidal composition, the filtrate may be present in an amount effective for maintaining a cell abundance of the dinoflagellate in the environment at no more than 50%, 60%, 70%, 80%, 90% or 95% of a cell abundance of the dinoflagellate in the environment treated with a control composition over a period of, for example, at least 1, 2, 3, 4, 5, 6, 7, 8, 9, 10, 11, 12, 13, 14, 15, 16, 17, 18, 19, 20, 21 or 28 days, or at least 1, 2, 3, 4, 5, 6, 7 or 8 weeks. In one embodiment, the second algicidal composition comprises the filtrate in an amount effective for maintaining a cell abundance of the dinoflagellate in the environment for at least 6 days at no more than 80% of a cell abundance of the dinoflagellate in the environment treated with a control composition, and the control composition comprises the matrix and the medium but not the filtrate.

In the second algicidal composition, the filtrate may be present in an amount effective for maintaining a growth rate of the dinoflagellate in the environment at no more than 50%, 60%, 70%, 80%, 90% or 95% of a growth rate of the dinoflagellate in the environment treated with a control composition over a period of, for example, at least 1, 2, 3, 4, 5, 6, 7, 8, 9, 10, 11, 12, 13, 14, 15, 16, 17, 18, 19, 20, 21 or 28 days, or at least 1, 2, 3, 4, 5, 6, 7 or 8 weeks. In one embodiment, the second algicidal composition comprises the filtrate in an amount effective for maintaining a growth of the dinoflagellate in the environment for at least 6 days at no more than 80% of a growth rate of the dinoflagellate in the environment treated with a control composition, and the control composition comprises the matrix and the medium but not the filtrate.

According to the second algicidal composition, the Shewanella strain IRI-160 culture may comprise the Shewanella strain IRI-160 at a concentration of at least 10¹⁰, 10⁹, 10⁸, 10⁷, 10⁶, 10⁵ or 10⁴ cells per mL. In one embodiment, the filtrate in the second algicidal composition may be prepared from a Shewanella strain IRI-160 culture containing the Shewanella strain IRI-160 at a concentration of at least 10⁸ cells per mL

In the second algicidal composition, the filtrate is algicidal. The second algicidal composition may retain a desirable percentage of the algicidal filtrate after storage. The algicidal composition may be stored at a temperature from 4° C. to 30° C. for a period of, for example, at least 1, 2, 3, 4, 5, 6, 7, 8, 9, 10, 11, 12, 13, 14, 15, 16, 17, 18, 19, 20, 21 or 28 days, or at least 1, 2, 3, 4, 5, 6, 7 or 8 weeks. At least 50%, 55%, 60%, 65%, 70%, 75%, 80%, 85%, 90% or 95% of the algicidal filtrate may be retained after the storage. In one embodiment, the second algicidal composition retains at least 80% of the algicidal filtrate after storage at 4° C. for at least 14 days.

The second algicidal composition may remain effective for inhibiting growth of a dinoflagellate in an environment after storage. The second algicidal composition may be stored at a temperature from 4° C. to 30° C. for a period of, for example, at least 1, 2, 3, 4, 5, 6, 7, 8, 9, 10, 11, 12, 13, 14, 15, 16, 17, 18, 19, 20, 21 or 28 days, or at least 1, 2, 3, 4, 5, 6, 7 or 8 weeks. The second algicidal composition may remain at least 50%, 55%, 60%, 65%, 70%, 75%, 80%, 85%, 90% or 95% effective for inhibiting growth of a dinoflagellate in an environment after storage. In one embodiment, the second algicidal composition remain effective for inhibiting growth of a dinoflagellate in an environment after storage at 4° C. for at least 14 days.

According to the first or second algicidal composition of the present invention, the dinoflagellate may be selected from the group consisting of genus Karlodinium, Gyrodiunium, Pfiesteria, Alexandrium, Cochlodinium, Dinophysis, Karenia, Prorocentrum, Heterocapsa, and Oxyrrhis. The dinoflagellate may be selected from the group consisting of species Pfiesteria piscicida, Karlodinium veneficum, Alexandrium fundyense, Alexandrium tamarense, Cochlodinium polykrikoides, Cochlodinium polykrikoides, Dinophysis acuminate, Gyrodinium instriatum, Gyrodinium uncatenum, Heterocapsa triquetra, Karenia brevis, Lavenderina fissa (aka Gyrodinium instriatum and Gyrodinium uncatenum), Oxyrrhis marina and Prorocentrum minimum.

According to the first or second algicidal composition of the present invention, the matrix may be porous. The matrix may have a porosity of at least 10%, 20%, 30%, 40%, 50%, 60%, 70% or 80%. In one embodiment, the matrix has a porosity of at least 50%. The matrix may comprise an agent capable of immobilizing Shewanella strain IRI-160 or a filtrate of a Shewanella strain IRI-160 culture. Suitable agents for making the matrix may be selected from the group consisting of alginate, agarose, cellulose, polyester and a combination thereof. The first algicidal composition may be in a form selected from the group consisting of alginate beads, agarose cubes, cellulosic sponge, polyester foam and combinations thereof. The second algicidal composition may be in a form selected from the group consisting of alginate beads, agarose cubes and combinations thereof. In one embodiment, the matrix in the first or second algicidal composition comprises alginate. In another embodiment, the algicidal composition is in the form of alginate beads, in which the Shewanella strain IRI-160 or the filtrate is immobilized for the first or the second algicidal composition, respectively.

According to the first or second algicidal composition of the present invention, the medium may be any medium suitable for maintaining algicidal effectiveness of the composition. In one embodiment, the medium may be a casein amino acid (CAA) medium comprising natural seawater, f/2 nutrients and casein amino acids. Examples of the f/2 nutrients include NaNO3, NaH2PO4 H2O, Na2CO3, trace metals, and vitamins (Guillard and Ryther, in Canadian Journal of Microbiology 8, 229-239 (1962)). The casein amino acids may be selected from the group consisting of casein enzymatic hydrolysate from bovine milk. The algicide composition may comprise the casein amino acids at a concentration between 0.5 g/L (1× concentration) and 5 g/L (10× centration).

The present invention provides a first preparation method. The first preparation method comprises immobilizing Shewanella strain IRI-160 in a medium to a matrix. A resulting algicidal composition is prepared, and the Shewanella strain IRI-160 is immobilized to the matrix in the resulting algicidal composition. The resulting algicidal composition comprises the Shewanella strain IRI-160 in an amount effective for inhibiting growth of a dinoflagellate in an environment. The Shewanella strain IRI-160 may be present in an amount effective for reducing a cell abundance or growth rate of the dinoflagellate in the environment as compared with a control composition. The control composition may be the same as the resulting algicidal composition except that the Shewanella strain IRI-160 is not immobilized to the matrix in the control composition. The Shewanella strain IRI-160 may be present in an amount effective for maintaining a cell abundance of the dinoflagellate in the environment at no more than 50%, 60%, 70%, 80%, 90% or 95% of a cell abundance of the dinoflagellate in the environment treated with a control composition over a period of, for example, at least 1, 2, 3, 4, 5, 6, 7, 8, 9, 10, 11, 12, 13, 14, 15, 16, 17, 18, 19, 20, 21 or 28 days, or at least 1, 2, 3, 4, 5, 6, 7 or 8 weeks. For example, the Shewanella strain IRI-160 may be present in an amount effective for maintaining a cell abundance of the dinoflagellate in the environment for at least 6 days at no more than 80% of a cell abundance of the dinoflagellate in the environment treated with a control composition, and the control composition comprises the matrix and the medium but not the Shewanella strain IRI-160. The Shewanella strain IRI-160 may be present in an amount effective for maintaining a growth rate of the dinoflagellate in the environment at no more than 50%, 60%, 70%, 80%, 90% or 95% of a growth rate of the dinoflagellate in the environment treated with a control composition over a period of, for example, at least 1, 2, 3, 4, 5, 6, 7, 8, 9, 10, 11, 12, 13, 14, 15, 16, 17, 18, 19, 20, 21 or 28 days, or at least 1, 2, 3, 4, 5, 6, 7 or 8 weeks. For example, the Shewanella strain IRI-160 may be present in an amount effective for maintaining a growth of the dinoflagellate in the environment for at least 6 days at no more than 80% of a growth rate of the dinoflagellate in the environment treated with a control composition, and the control composition comprises the matrix and the medium but not the Shewanella strain IRI-160. The resulting composition may comprise the Shewanella strain IRI-160 at a concentration of at least 10¹⁰, 10⁹, 10⁸, 10⁷, 10⁶, 10⁵ or 10⁴ cells per mL. For example, the Shewanella strain IRI-160 may be present at a concentration of at least 10⁸ cells per mL. The resulting algicidal composition may retain a desirable percentage of the algicidal Shewanella strain IRI-160 after storage. The resulting algicidal composition may be stored at a temperature from 4° C. to 30° C. for a period of, for example, at least 1, 2, 3, 4, 5, 6, 7, 8, 9, 10, 11, 12, 13, 14, 15, 16, 17, 18, 19, 20, 21 or 28 days, or at least 1, 2, 3, 4, 5, 6, 7 or 8 weeks. At least 50%, 55%, 60%, 65%, 70%, 75%, 80%, 85%, 90% or 95% of the algicidal Shewanella strain IRI-160 may be retained after the storage. For example, at least 80% of the algicidal Shewanella strain IRI-160 may be retained after the resulting algicidal composition is stored at 4° C. for at least 14 days.

The present invention provides a second preparation method. The second preparation method comprises immobilizing a filtrate of a Shewanella strain IRI-160 culture in a medium to a matrix. A resulting algicidal composition is prepared, and the filtrate is immobilized to the matrix in the resulting algicidal composition. The resulting algicidal composition comprises the filtrate in an amount effective for inhibiting growth of a dinoflagellate in an environment. The filtrate may be present in an amount effective for reducing a cell abundance or growth rate of the dinoflagellate in the environment as compared with a control composition. The control composition may be the same as the resulting algicidal composition except that the filtrate is not immobilized to the matrix in the control composition. The filtrate may be present in an amount effective for maintaining a cell abundance of the dinoflagellate in the environment at no more than 50%, 60%, 70%, 80%, 90% or 95% of a cell abundance of the dinoflagellate in the environment treated with a control composition over a period of, for example, at least 1, 2, 3, 4, 5, 6, 7, 8, 9, 10, 11, 12, 13, 14, 15, 16, 17, 18, 19, 20, 21 or 28 days, or at least 1, 2, 3, 4, 5, 6, 7 or 8 weeks. For example, the filtrate may be present in an amount effective for maintaining a cell abundance of the dinoflagellate in the environment for at least 6 days at no more than 80% of a cell abundance of the dinoflagellate in the environment treated with a control composition, and the control composition comprises the matrix and the medium but not the filtrate. The filtrate may be present in an amount effective for maintaining a growth rate of the dinoflagellate in the environment at no more than 50%, 60%, 70%, 80%, 90% or 95% of a growth rate of the dinoflagellate in the environment treated with a control composition over a period of, for example, at least 1, 2, 3, 4, 5, 6, 7, 8, 9, 10, 11, 12, 13, 14, 15, 16, 17, 18, 19, 20, 21 or 28 days, or at least 1, 2, 3, 4, 5, 6, 7 or 8 weeks. For example, the filtrate may be present in an amount effective for maintaining a growth of the dinoflagellate in the environment for at least 6 days at no more than 80% of a growth rate of the dinoflagellate in the environment treated with a control composition, and the control composition comprises the matrix and the medium but not the filtrate. The Shewanella strain IRI-160 culture may comprise Shewanella strain IRI-160 at a concentration of at least 10¹⁰, 10⁹, 10⁸, 10⁷, 10⁶, 10⁵ or 10⁴ cells per mL, for example, at least 10⁸ cells per mL. The resulting algicidal composition may retain a desirable percentage of the algicidal filtrate after storage. The resulting algicidal composition may be stored at a temperature from 4° C. to 30° C. for a period of, for example, at least 1, 2, 3, 4, 5, 6, 7, 8, 9, 10, 11, 12, 13, 14, 15, 16, 17, 18, 19, 20, 21 or 28 days, or at least 1, 2, 3, 4, 5, 6, 7 or 8 weeks. At least 50%, 55%, 60%, 65%, 70%, 75%, 80%, 85%, 90% or 95% of the algicidal filtrate may be retained after the storage. For example, at least 80% of the algicidal filtrate may be retained after the resulting algicidal composition is stored at 4° C. for at least 14 days.

According to the first or second preparation method of the present invention, the dinoflagellate may be selected from the group consisting of genus Karlodinium, Gyrodiunium, Pfiesteria, Alexandrium, Cochlodinium, Dinophysis, Karenia, Prorocentrum, Heterocapsa, and Oxyrrhis. The dinoflagellate may be selected from the group consisting of species Pfiesteria piscicida, Karlodinium veneficum, Alexandrium fundyense, Alexandrium tamarense, Cochlodinium polykrikoides, Cochlodinium polykrikoides, Dinophysis acuminate, Gyrodinium instriatum, Gyrodinium uncatenum, Heterocapsa triquetra, Karenia brevis, Lavenderina fissa (aka Gyrodinium instriatum and Gyrodinium uncatenum), Oxyrrhis marina and Prorocentrum minimum.

According to the first or second preparation method, the matrix may be porous. The matrix may have a porosity of at least 10%, 20%, 30%, 40%, 50%, 60%, 70% or 80%. In one embodiment, the matrix has a porosity of at least 50%. The matrix may comprise an agent capable of immobilizing Shewanella strain IRI-160 or a filtrate of the Shewanella strain IRI-160 culture. Suitable agents for making the matrix may be selected from the group consisting of alginate, agarose, cellulose, polyester and a combination thereof. The first algicidal composition may be in a form selected from the group consisting of alginate beads, agarose cubes, cellulosic sponge, polyester foam and combinations thereof. The second algicidal composition may be in a form selected from the group consisting of alginate beads, agarose cubes and combinations thereof. In one embodiment, the matrix in the first or second algicidal composition comprises alginate. In another embodiment, the algicidal composition is in the form of alginate beads, in which the Shewanella strain IRI-160 or the filtrate is immobilized for the first or the second algicidal composition, respectively.

According to the first or second preparation method, the medium may be any medium suitable for maintaining algicidal effectiveness of the composition. In one embodiment, the medium may be a casein amino acid (CAA) medium comprising natural seawater, f/2 nutrients and casein amino acids. Examples of the f/2 nutrients include NaNO3, NaH2PO4 H2O, Na2CO3, trace metals, and vitamins (Guillard and Ryther, in Canadian Journal of Microbiology 8, 229-239 (1962)). The casein amino acids may be selected from the group consisting of casein enzymatic hydrolysate from bovine milk. The algicide composition may comprise the casein amino acids at a concentration between 0.5 g/L (1× concentration) and 5 g/L (10× centration).

The present invention further provides a first treatment method for inhibiting growth of a dinoflagellate in an environment. The first treatment method comprises applying an effective amount of an algicidal composition to the dinoflagellate in the environment. The algicidal composition comprises Shewanella strain IRI-160, a matrix and a medium and the Shewanella strain IRI-160 is immobilized to the matrix. The first treatment method may further comprise maintaining a cell abundance of the dinoflagellate in the environment for at least 6 days at no more than 80% of a cell abundance of the dinoflagellate in the environment treated with a control composition. The control composition may comprise the matrix and the medium but not the Shewanella strain IRI-160. The first treatment method may further comprise storing the algicidal composition at a temperature of 4° C. for at least 14 days before applying the composition.

The present invention further provides a second treatment method for inhibiting growth of a dinoflagellate in an environment. The second treatment method comprises applying an effective amount of an algicidal composition to the dinoflagellate in the environment. The algicidal composition comprises a filtrate of a Shewanella strain IRI-160 culture, a matrix and a medium and the filtrate is immobilized to the matrix. The second treatment method may further comprise maintaining a cell abundance of the dinoflagellate in the environment for at least 6 days at no more than 80% of a cell abundance of the dinoflagellate in the environment treated with a control composition. The control composition may comprise the matrix and the medium but not the filtrate. The second treatment method may further comprise storing the algicidal composition at a temperature of 4° C. for at least 14 days before applying the composition.

According to the first or second treatment method of the present invention, the dinoflagellate may be selected from the group consisting of genus Karlodinium, Gyrodiunium, Pfiesteria, Alexandrium, Cochlodinium, Dinophysis, Karenia, Prorocentrum, Heterocapsa, and Oxyrrhis. The dinoflagellate may be selected from the group consisting of species Pfiesteria piscicida, Karlodinium veneficum, Alexandrium fundyense, Alexandrium tamarense, Cochlodinium polykrikoides, Cochlodinium polykrikoides, Dinophysis acuminate, Gyrodinium instriatum, Gyrodinium uncatenum, Heterocapsa triquetra, Karenia brevis, Lavenderina fissa (aka Gyrodinium instriatum and Gyrodinium uncatenum), Oxyrrhis marina and Prorocentrum minimum.

According to the first or second treatment method of the present invention, the matrix may be porous. The matrix may have a porosity of at least 10%, 20%, 30%, 40%, 50%, 60%, 70% or 80%. In one embodiment, the matrix has a porosity of at least 50%. The matrix may comprise an agent capable of immobilizing Shewanella strain IRI-160 or a filtrate of the Shewanella strain IRI-160 culture. Suitable agents for making the matrix may be selected from the group consisting of alginate, agarose, cellulose, polyester and a combination thereof. The first algicidal composition may be in a form selected from the group consisting of alginate beads, agarose cubes, cellulosic sponge, polyester foam and combinations thereof. The second algicidal composition may be in a form selected from the group consisting of alginate beads, agarose cubes and combinations thereof. In one embodiment, the matrix in the first or second algicidal composition comprises alginate. In another embodiment, the algicidal composition is in the form of alginate beads, in which the Shewanella strain IRI-160 or the filtrate is immobilized for the first or the second algicidal composition, respectively.

According to the first or second treatment method of the present invention, the medium may be any medium suitable for maintaining algicidal effectiveness of the composition. In one embodiment, the medium may be a casein amino acid (CAA) medium comprising natural seawater, f/2 nutrients and casein amino acids. Examples of the f/2 nutrients include NaNO3, NaH2PO4 H2O, Na2CO3, trace metals, and vitamins (Guillard and Ryther, in Canadian Journal of Microbiology 8, 229-239 (1962)). The casein amino acids may be selected from the group consisting of casein enzymatic hydrolysate from bovine milk. The algicide composition may comprise the casein amino acids at a concentration between 0.5 g/L (1× concentration) and 5 g/L (10× centration).

### EXAMPLE 1. IMMOBILIZED SHEWANELLA SP. IRI-160 AND APPLICATION THEREOF

Shewanella sp. IRI-160 is an algicidal bacterium isolated from Delaware Inland Bays. It secretes water-soluble compounds that inhibit the growth of dinoflagellates. Previous research indicated that this bacterium does not have a negative impact on other algal species. In this research, Shewanella sp. IRI-160 was immobilized to different porous matrices, including agarose, alginate hydrogel, cellulosic sponge, and polyester foam. The retention of Shewanella sp. IRI-160 on or within these matrices was examined at 4 and 25° C. for 12 days. Results indicated that alginate was superior in terms of cell retention, with >99% of Shewanella cells retained in the matrix after 12 days. Shewanella sp. IRI-160 cells were then immobilized within alginate beads to evaluate algicidal effects on harmful dinoflagellates Karlodinium veneficum and Prorocentrum minimum at bacterial concentrations of 10⁶ to 10⁸ cells mL⁻¹. The effects on dinoflagellates were compared to non-harmful cryptophyte Rhodomonas sp., as well as the effects of free-living bacteria on these species. Results indicated that immobilized Shewanella sp. IRI-160 in alginate beads were as effective as the free-living bacteria to control the growth of K. veneficum and P. minimum, while no negative impacts of immobilized Shewanella sp. IRI-160 on the non-harmful control species Rhodomonas sp. were observed. Overall, this study suggests that immobilized Shewanella sp. IRI-160 may be used as an environmentally friendly approach to prevent or mitigate the blooms of harmful dinoflagellates and provides insight and directions for future studies.

### 1. Material and Methods

### 1.1. Attachment and Immobilization of Shewanella Sp. IRI-160

### 1.1.1. Bacterium Culture Preparation

Cultures of Shewanella sp. IRI-160 were prepared as described (Pokrzywinski et al., in Harmful Algae 19:23-29 (2012)). Briefly, Shewanella sp. IRI-160 was transferred from a single colony into liquid LM medium (Sambrook et al., in Molecular cloning: a laboratory manual. Cold spring harbor laboratory press, 1989) and incubated overnight at 25° C. with shaking at 100 rpm. The optical density of bacterial cultures was measured on the following day, and cultures were diluted with liquid LM medium to an OD₆₀₀ of 1.4 before use.

### 1.1.2 Immobilization and Retention of Bacteria in Alginate Beads

The methods of immobilizing Shewanella sp. IRI-160 into different matrices were modified from Kang et al. (2007). A 2% (w/v) solution of sterile alginic acid, sodium salt (catalog #177772500; Thermofisher Acros Organics, Belgium) in MilliQ water was mixed with diluted bacterial culture with a 5:1 (v/v) ratio of alginate: bacterial culture. To make alginate beads, the mixture was extruded from a sterile syringe through sterile silicone tubing into a beaker of cold sterile 0.2 M CaCl₂ solution. The solution in the beaker was mixed at low speed during extrusion. The resultant alginate beads were approximately 5 mm in diameter. The beads were washed using sterile f/2 medium (—Si and a salinity of 20; Guillard and Ryther, in Canadian Journal of Microbiology 8, 229-239 (1962) twice and kept in f/2 medium before use on the same day. Prior to the start of the experiment, beads were transferred into f/2 medium (total volume of alginate beads: total volume of medium=1:10) and divided into 2 groups (N=3). One group was incubated at 4° C. and another group was incubated at 25° C.

On Days 0, 3, 6, and 12, an aliquot of the medium surrounding the beads from each replicate was collected for cell counts using the method described below. Beads were also removed and dissolved in sterile 1% (w/v) sodium pyrophosphate in MilliQ water to release the bacteria, and cells were counted as below.

### 1.1.3. Immobilization and Retention of Bacteria in Agarose Cubes

Six percent (w/v) sterile low-melting agarose (catalog #S209-500; Fisher Scientific, Pittsburgh, Pa.) in MilliQ water was heated by microwave, cooled to room temperature and mixed with the bacterial culture at a ratio of 10:1 (v/v). The mixture was poured into sterile Petri plates to a thickness of 5 mm. The mixture in the covered Petri plates was allowed to solidify and then cut into small cubes of 5×5×5 mm³ using sterile scalpels. The cubes were washed and prepared as described above (Section 1.1.2).

On Days 0, 3, 6, and 12, bacterial cells in the surrounding media were counted as below. Cubes from each replicate were then transferred into sodium acetate buffer (pH 4.6; 49% 0.2 M sodium acetate [Fisher Scientific], 51% 0.2 M acetic acid [Fisher Scientific]), and heated at 90° C. for 10 min. Bacterial cells released from the agarose matrix were counted as described below.

### 1.1.4. Immobilization and Retention of Bacteria in the Cellulosic Sponge and Polyester Foam

Cellulosic sponge (3M, Maplewood, Minn.) and polyester foam (Danner Mfg. Inc., Islandia, N.Y.) were cut into small cubes with volumes of approximately 0.125 cm³ and 1 cm³, respectively. Sponge and polyester cubes were autoclaved and each added to the diluted bacterial culture at a ratio of 1:10 (v/v). The mixture was incubated at 25° C., with shaking at 100 rpm for 3 hours, and then incubated at room temperature without shaking overnight. Cellulosic sponge and polyester cubes were washed using sterile f/2 medium twice and kept in f/2 medium before use on the same day. Cubes were washed and the experiment was prepared as described above (Section 1.1.2).

On Days 0, 3, 6, and 12, an aliquot of the medium surrounding the cubes from each replicate was collected for cell counts as described below. Cubes were transferred from each replicate into acetate buffer (49% 0.2M sodium acetate, 51% 0.2M acetic acid, pH 4.6) and sonicated using an ultrasonic bath (Fisher Scientific) for 15 min at 25° C. to release the bacteria. The cell suspension was diluted 1:10 with acetate buffer for cell counts.

### 1.1.5. Free-Living Bacteria Preparation

The bacterial suspension was prepared by centrifuging bacterial culture in LM medium at 6000 rpm for 5 min. The supernatant was discarded and bacteria were washed with f/2 medium, and then resuspended in f/2 medium before use on the same day. The bacterial suspension in f/2 medium was divided into two groups and incubated at 4 and 25° C. as above (N=3). On Days 0, 3, 6, and 12, one milliliter of free-living bacteria from each treatment was removed and diluted to 1:100 for cell counts as described below.

### 1.1.6. Bacterial Cell Counts

Bacterial cell counts were performed. Bacteria were fixed with 1.7% formaldehyde (v/v) and stained with sterile 0.1 mg mL⁻¹ DAPI (4′,6-diamidino-2-phenylindole, dilactate; ThermoFisher Scientific, Waltham, Mass., USA). Stained cells were filtered onto 0.2 μm black polycarbonate filters (Millipore, Bedford, Mass., USA). Filters were preserved with mountant solution (ElectronMicroscopy Sciences, Hatfield, Pa., USA) on glass slides and cells were counted using a fluorescent microscope (EVOS® FL Auto Imaging System; ThermoFisher Scientific) equipped with a DAPI light cube (excitation: 357/44 nm, emission: 447/60 nm; ThermoFisher Scientific). Cells were counted at a magnification of 100×; at least 3 fields were counted for each sample. Cell density was calculated as follows:

Cells mL⁻¹ =(membrane conversion factor)(avg. number of bacteria per micrometer field)(dilution factor)⁻¹

where the membrane conversion factor is the filtration area divided by the area of the micrometer field.

### 1.2. Algicidal Effect of Immobilized Shewanella sp. IRI-160 in Alginate Beads

### 1.2.1. Algal Stock Cultures

Stock cultures of Karlodinium veneficum (CCMP 2936 [National Center for Marine Algae and Microbiota, https://ncma.bigelow.org/]), Prorocentrum minimum (CCMP2233), and control species Rhodomonas sp. (CCMP 757; cryptophyte) were cultured in natural seawater at 25° C. with f/2 nutrients (—Si; Guillard and Ryther 1962) and a salinity of 20, with a light intensity of approximately 130 μmol photons m⁻² s⁻¹. Cultures were kept under a 12 h: 12 h light: dark cycle, and semi-continuously in the exponential growth phase. Stock cultures were diluted to a cell density of 54,000 to 59,000 cells mL⁻¹ prior to the start of each experiment.

### 1.2.2. Bacterial Culture Preparation

Preliminary results indicated that LM medium may have negative effects on laboratory algal cultures, but that laboratory cultures were able to grow with low concentrations [0.05% (w/v)] of casein amino acids (data not shown). For experiments with algae, Shewanella sp. IRI-160 was cultured with sterile 0.05% [w/v] casein amino acids (CAA; Sigma-Aldrich, St. Louis, Mo., USA) in f/2 medium (CAA medium) to avoid the negative impacts of bacterial growth medium on algae. A single colony of Shewanella sp. IRI-160 from LM plates was transferred to 2 g L⁻¹ CAA medium, and the bacterial culture was incubated overnight as described above. The bacterial culture was then diluted 1:4 with f/2 medium and incubated at room temperature without shaking overnight.

### 1.2.3. Preparation of Immobilized Bacteria in Alginate Beads

Sterile 4% (w/v) alginic acid, sodium salt (Thermofisher Acros Organics) in 0.5 g L⁻¹ CAA medium was mixed with Shewanella sp. IRI-160 culture in a ratio of 1:1 (v/v). The alginate beads were extruded using the same method as above into cold 0.4 mol L⁻¹ CaCl₂. Control beads were prepared using the same process with the addition of sterile CAA medium but without the addition of bacteria. Alginate beads were stored in 0.5 g L⁻¹ CAA medium at 4° C. before use within 2 weeks. Immobilized bacteria cell densities were determined as above before the start of the experiment.

### 1.2.4. Effects of Immobilized Bacteria on Dinoflagellates

Alginate beads with immobilized bacteria were added to cultures of K. veneficum, P. minimum, and Rhodomonas sp. in 250 mL polycarbonate flasks to achieve 10⁶, 10⁷, and 10⁸ cells mL⁻¹ of bacteria in algal cultures (N=3). Additional beads, without bacteria, were also added to the treatments in the 10⁶ and 10⁷ cells mL⁻¹ groups so that all treatments would have the same number of beads as the 10⁸ cells mL⁻¹ treatment group. For comparison, free bacteria (not immobilized) were added to separate cultures (N=3) to reach final bacterial concentrations of 10⁸ cells mL⁻¹. CAA medium was also added to the cultures in the free-living bacteria treatment group to achieve the same concentrations of CAA medium as in other treatments. Finally, in the control group, control beads were added to cultures (N=3) at the same bead density as in the treatment groups.

Algal cultures were incubated under the same condition as stock cultures. In vivo fluorescence was measured at the beginning of the experiment, and then after 1, 2, 4 and 6 days. Specific growth rates (μ) were calculated over 6 days as (Guillard et al. 1973):

$$μ = \frac{ln ( N 2 / N 1 )}{T 2 - T 1}$$

where N2 is the in vivo fluorescence reading of each culture at T2 (Day 6); N1 is the average in vivo fluorescence of each species at T1 (Day 0, of the bulk cultures). Algal cell density was also determined by cell counts at the initial time point and on the last day.

Bacteria associated with alginate beads in the control group (contributed from non-axenic algal cultures) and the 10⁸ cells mL⁻¹ treatment group were counted using the methods described above at the termination of the experiment. Additionally, on the last day, cultures were filtered through 3.0 μm polycarbonate filters (Millipore) and then onto 0.2 μm black polycarbonate filters (Millipore) from each treatment to determine total free-living bacterial cell densities in each culture as described above. For comparison, cell densities of bacteria in each group were divided by average cell densities of controls to calculate the “relative bacterial cell densities”. An aliquot of each sample was also filtered through 0.2 μm nylon syringe filters (Corning, Corning, N.Y., USA) to measure ammonium concentrations using the procedure described below.

### 1.2.5 Ammonium Concentration

Ammonium concentrations were measured using an API® Ammonia Test Kit (Mars Fishcare Inc., Chalfont, Pa., US) modified as described here. Briefly, each of 250 μL hypochlorite solution and salicylate/catalyst solution from the kit were added to 2.5 mL of diluted samples (with sterile MilliQ water), and samples were incubated at room temperature for 10 min for the development of color. Absorbance at 690 nm was measured (NanoDrop 2000 Spectrophotometer; ThermoFisher Scientific) and the concentration of ammonium in each sample was determined by linear regression analysis using a standard curve of ammonium standards (Sigma-Aldrich) ranging from 0 to 200 μM (in MilliQ water).

### 1.2.6. Statistical Analyses

Repeated measures ANOVA was used to test if there was a significant difference in cell densities of Shewanella sp. IRI-160 immobilized to each matrix over time, and was also used to test if the densities of free-living Shewanella sp. IRI-160 and Shewanella sp. IRI-160 released into the medium changed significantly over time (p<0.05). If there was a significant difference, then paired t-test was used to analyze the significant difference in cell densities between all pairs of time points (p<0.05). One-way ANOVA was used to test the significant impacts of temperature on the densities of immobilized and free-living Shewanella sp. IRI-160, as well as Shewanella sp. IRI-160 released to the surrounding medium from each matrix at each time point (p<0.05).

In addition, repeated measures ANOVA was conducted to test the significant difference in in vivo fluorescence of each algal species in control and treatment groups over time (p<0.05). One-way ANOVA was used to test the significant difference in in vivo fluorescence of each algal species between groups at each time point, as well as the specific growth rates between groups of each species (p<0.05). If a significant difference was detected, then Tukey HSD test was used to analyze the significant difference in in vivo fluorescence of each algal species between all pairs of groups at each time point, as well as the specific growth rates of each species between all pairs of groups (p<0.05). One-way ANOVA was also used to test the significant differences in cell densities of each algal species between groups on Day 6, and if a significant difference was detected, then Tukey HSD test was conducted to test the significant difference between all pairs of groups (p<0.05).

A paired t-test was used to measure the significant difference in densities of immobilized bacterial cells in alginate beads between Day 0 and Day 6 in each algal culture treated with 10⁸ cells mL⁻¹ immobilized Shewanella sp. IRI-160 (p<0.05); one-sample t-test was used to measure if true means of bacterial cell densities in alginate beads in control cultures on Day 6 were 0 (to measure the significant difference between these cell densities and 0; p<0.05). One-way ANOVA was used to test for significant differences in relative densities of free-living bacteria in treatments and controls on Day 6 in each group of algal cultures, as well as absolute and relative ammonium concentrations of these cultures on the same time point (p<0.05); if a significant difference was detected, Tukey HSD test was conducted to test the significant difference between all pairs of groups (p<0.05). All statistical analyses were conducted using R (v. 3.6.0; R Core Team, 2015).

### 2. Results

### 2.1. Retention of Immobilized Shewanella Sp. IRI-160

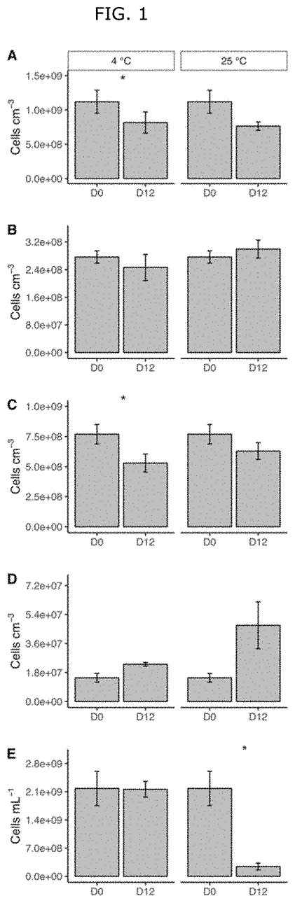

The distribution of Shewanella sp. IRI-160 cells that were in each matrix vs. those in the surrounding medium after 12 days was assessed at 4 and 25° C. (FIG. 1A-D; Table 1). When immobilized in alginate beads at 25° C., 99.83% of total Shewanella sp. IRI-160 cells counted were within the matrix on Day 12, while 0.17% of the cells were not associated with the matrix and were in the surrounding medium (Table 1). At 4° C., 99.94% of cells counted were in alginate beads on Day 12 and 0.06% of the cells were in the surrounding medium. When immobilized in agarose cubes, 84.09% and 81.05% of cells counted were in the matrix at 25 and 4° C., respectively. Note that data for Shewanella sp. IRI-160 immobilized in agarose cubes at 25° C. on Day 12 were excluded from statistical analyses because one replicate was lost due to contamination. Furthermore, 98.92% and 98.94% of cells counted on Day 12 were in the sponge cubes at 25 and 4° C., respectively. For polyester, 96.55% and 92.97% of cells counted on Day 12 were immobilized within the polyester cubes at 25 and 4° C., respectively (FIG. 1, Table 1).

### 2.1.1. Effects of Temperature on Immobilized Shewanella Sp. IRI-160

The density of immobilized Shewanella sp. IRI-160 decreased slightly but significantly over the 12-day period in alginate beads and sponge at 4° C. (p<0.05) but not 25° C. (FIGS. 1A and C). No significant changes over time were observed for the density of immobilized Shewanella sp. IRI-160 in agarose cubes or polyester at either temperature (p>0.05; FIGS. 1B and D). There was no significant impact of temperature on cell densities of Shewanella sp. IRI-160 retained in alginate beads, agarose, or sponge cubes at any time point tested (p>0.05; FIG. 1). Cell densities of Shewanella sp. IRI-160 immobilized in polyester cubes at 25° C., however, were >2-fold higher than those at 4° C. on Day 12 (p<0.05; FIG. 1D).

In contrast to the relatively stable densities of Shewanella sp. IRI-160 immobilized to the four matrices, the cell density of Shewanella sp. IRI-160 in the free-living treatment group (not immobilized within a matrix) decreased significantly at 25° C. (p<0.05; FIG. 1E), but not at 4° C. In addition, the cell density of free-living Shewanella sp. IRI-160 at 4° C. was significantly higher than at 25° C. on Day 12 (p<0.05).

### 2.2. Effects of Immobilized Shewanella Sp. IRI-160 on Algae

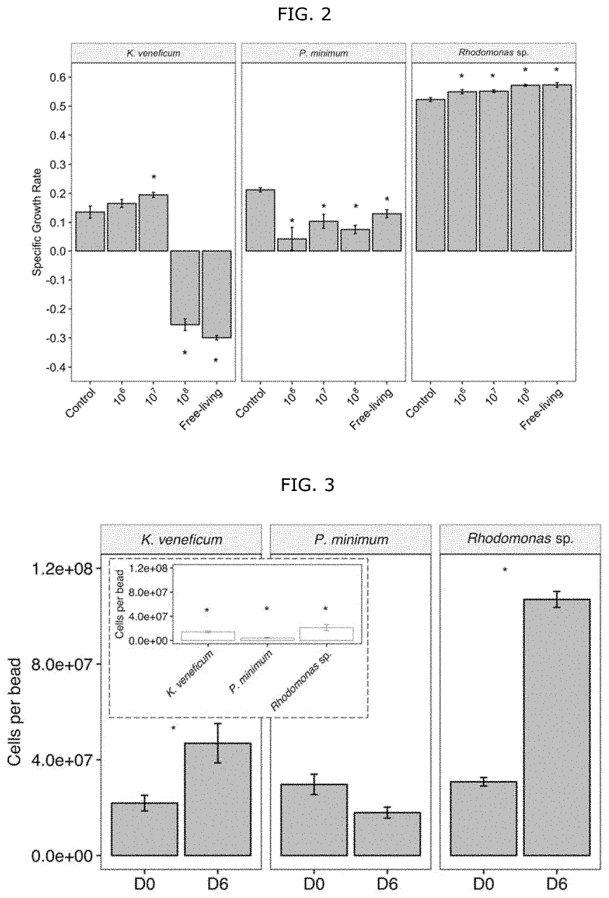

Shewanella sp. IRI-160 was immobilized in alginate beads, which were then added to achieve densities of 10⁶ to 10⁸ cells mL⁻¹ in cultures of harmful dinoflagellates K. veneficum (FIG. 2; FIG. 6A; Table 2) and P. minimum (FIG. 2; FIG. 6B; Table 2), as well as the non-harmful cryptophyte Rhodomonas sp. (FIG. 2; FIG. 6C; Table 2). The effects of immobilized bacteria on algal growth were then evaluated over 6 days and compared to the effects of free-living Shewanella sp. IRI-160 and blank alginate beads with no bacteria (control). Cell densities based on microscopic cell counts were consistent with in vivo fluorescence of all species in cultures treated with immobilized or free-living Shewanella sp. IRI-160 (data not shown).

### 2.2.1 Effects of Immobilized and Free-Living Shewanella Sp. IRI-160 on Harmful Dinoflagellate K. veneficum

Growth rates of K. veneficum cultures were positive for control cultures as well as the 10⁶ and 10⁷ cells mL⁻¹ immobilized bacteria treatments over 6 days (FIG. 2). There were no significant differences in K. veneficum growth rates between the 10⁶ cells mL⁻¹ immobilized bacteria treatment and controls over the 6-day incubation period (p>0.05). K. veneficum growth in the 10⁷ cells mL⁻¹ immobilized bacteria treatment, however, was 1.44 times greater than the controls. In contrast to other treatments, the specific growth rates of cultures treated with 10⁸ cells mL⁻¹ immobilized and free-living Shewanella sp. IRI-160 were negative and significantly lower than other treatments and controls over the 6-day incubation period (p<0.05, FIG. 6A); in vivo fluorescence of these two treatments were also significantly lower than other groups at all time points tested (p<0.05). The in vivo fluorescence of K. veneficum cultures treated with 10⁸ cells mL⁻¹ immobilized bacteria was not significantly different from treatments with free-living bacteria at any time point tested (p>0.05, FIG. 6A), while the overall growth rate of K. veneficum in the 10⁸ cells mL⁻¹ immobilized bacteria was slightly but significantly higher in the immobilized bacteria compared to the free-living bacteria treatment (by 1.18 times; p<0.05).

### 2.2.2. Effects of Immobilized and Free-Living Shewanella Sp. IRI-160 on Harmful Dinoflagellate P. minimum

In contrast to K. veneficum, cultures of P. minimum had positive specific growth rates in all treatments and controls over 6 days (FIG. 2). However, the specific growth rate of P. minimum control cultures was significantly higher than the 10⁶, 10⁷, 10⁸ cells mL⁻¹ immobilized, and free-living bacteria treatments (p<0.05). In addition, the specific growth rate of P. minimum in the free-living bacteria treatment was significantly higher than the 10⁶ cells mL⁻¹ immobilized Shewanella sp. IRI-160 treatments (p<0.05), while no significant difference was observed between all other groups (p>0.05). Similar to K. veneficum, significant differences in in vivo fluorescence of P. minimum cultures were observed between controls and the 10⁸ cells mL⁻¹ or free-living bacteria treatments on Day 1 (p<0.05; FIG. 6B; Table 2).

### 2.2.3. Effects of Immobilized and Free-Living Shewanella Sp. IRI-160 on Non-Harmful Cryptophyte Rhodomonas Sp

The specific growth rates of the cryptophyte Rhodomonas sp. were slightly but significantly higher in all treatments compared to controls over the 6-day incubation period (p<0.05), with the highest values observed for cultures treated with the highest density of Shewanella sp. IRI-160 (FIG. 2). No significant differences in specific growth rates were observed between the free-living and 10⁸ cells mL⁻¹ immobilized bacteria treatments, or between the treatments with 10⁶ and 10⁷ cells mL⁻¹ immobilized bacteria.

### 2.2.4. Immobilized and Free-Living Bacterial Densities in Algal Cultures

There was a significant 2.14-fold increase in the density of immobilized bacteria (p<0.05) in the 10⁸ cells mL⁻¹ bacteria treatment by Day 6 in cultures of K. veneficum, and a 3.49-fold increase in the density of immobilized bacteria in cultures of Rhodomonas sp., but no significant increase in the density of immobilized bacteria in the same treatments of P. minimum (p>0.05; FIG. 3). In addition, the bacterial abundance in alginate beads without bacteria in the non-axenic control cultures of K. veneficum, P. minimum, and Rhodomonas sp. ranged from 4.15×10⁶ to 2.13×10⁷ cells per bead on Day 6 (FIG. 3, insert).

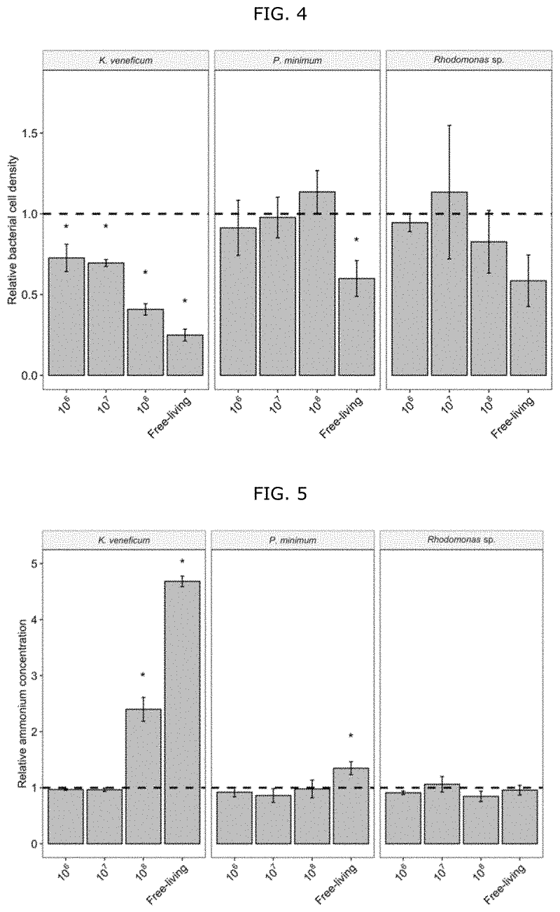

There were significant differences between cell densities of free-living bacteria in controls and treatments of K. veneficum and P. minimum on Day 6 (p<0.05), while there was no significant difference in Rhodomonas sp. cultures (p>0.05; FIG. 4). In K. veneficum cultures, the cell densities of free-living bacteria in controls were significantly higher than treatments, where free-living bacteria densities decreased with an increase in immobilized bacteria treatment (p<0.05). For P. minimum, the total abundance of free-living bacteria in controls was significantly higher than the free-living Shewanella sp. IRI-160 treatment (p<0.05), but not significantly different from the abundance of free-living bacteria in the 10⁸, 10⁷, and 10⁶ cells mL⁻¹ immobilized Shewanella sp. IRI-160 treatments.

### 2.2.5. Ammonium Concentration

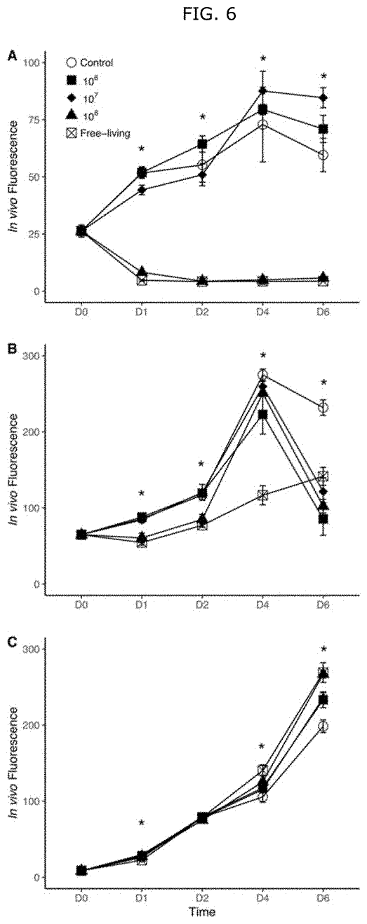

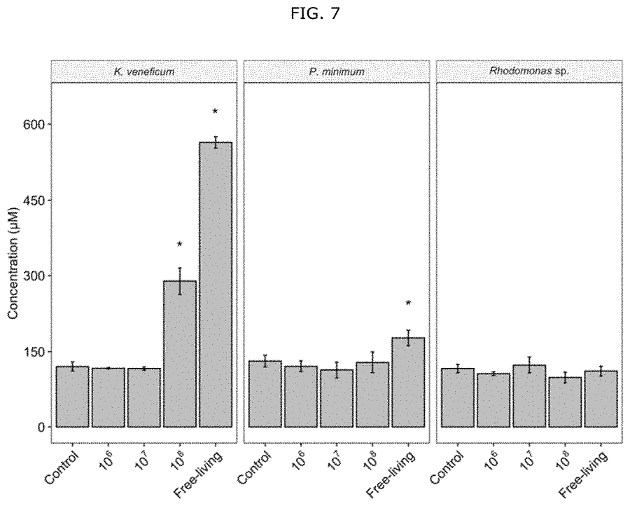

On Day 6, ammonium concentrations in algal cultures ranged from 98.2 to 565 μM (FIG. 7). Ammonium concentrations in K. veneficum cultures treated with free-living Shewanella sp. IRI-160 or with 10⁸ cells mL⁻¹ immobilized bacteria were significantly higher than controls as well as the 10⁶ and 10⁷ cells mL⁻¹ immobilized bacteria treatments (p<0.05; FIG. 5; FIG. 7) p<0.05). The ammonium concentration in free-living bacteria treatments was significantly higher than the cultures treated with 10⁸ cells mL⁻¹ immobilized Shewanella sp. IRI-160 (p<0.05). There were no significant differences in ammonium concentrations between other pairs of groups (p>0.05). Similarly, ammonium concentrations in cultures of P. minimum treated with free-living Shewanella sp. IRI-160 were significantly higher than controls and other treatments (p<0.05), while there was no significant difference between other pairs of groups (p>0.05; FIG. 5; FIG. 7). There was no significant difference in ammonium concentrations between controls and treatments of Rhodomonas sp. (p>0.05).

### 3. Discussion

Previous research indicated that bacterium Shewanella sp. IRI-160 and its water-soluble algicide IRI-160AA were able to control the growth of dinoflagellates with no negative impacts on cell densities of other phytoplankton species or organisms at higher trophic levels tested. Here, Shewanella sp. IRI-160 was immobilized using different porous matrices, including alginate hydrogel beads, as well as agarose, sponge, and polyester cubes. The retention of Shewanella sp. IRI-160 in each matrix was evaluated for a course of 12 days; the abundance of immobilized bacteria within each matrix was compared to the abundance of bacteria released into the surrounding medium, as well as to the density of bacteria that were not immobilized. To investigate the effect of temperature on immobilized Shewanella sp. IRI-160, each experiment was performed at 4 and 25° C. Alginate hydrogel beads were selected for further experiments due to its high retention of Shewanella sp. IRI-160, as well as its low-cost, non-toxic, and biodegradable characteristics. Shewanella sp. IRI-160 was immobilized in alginate hydrogel beads at three concentrations to evaluate the ability of immobilized Shewanella sp. IRI-160 to control the growth of harmful dinoflagellates.

Results of this research indicated that there was no or only a slight decrease in cell densities of free-living and immobilized Shewanella sp. IRI-160 at 4° C. (FIG. 1), consistent with previous studies demonstrating that growth at low temperatures (˜4° C.) is a hallmark of the Shewanella genus. Its survival at low temperatures may be related to changes in morphology as well as the production of alternative proteins and lipids. Additionally, there was a dramatic and significant decrease in cell densities of free-living Shewanella sp. IRI-160 at 25° C. (FIG. 1E), while the density of immobilized bacteria did not decrease significantly at the same temperature in any matrices tested (FIG. 1A-D), suggesting immobilization to matrices may provide advantages to these bacteria and protect them from environmental conditions where their free-living counterparts may not survive. Warmer temperatures are favorable for dinoflagellate blooms; and have been associated with their higher annual growth rate and longer duration of bloom seasons. The decrease of cell densities of free-living Shewanella sp. IRI-160 at 25° C. may limit its ability to control the growth of dinoflagellates in the environment, while immobilization to matrices may contribute to its long-lasting performance in the field.

Over the entire experiment period, the majority of Shewanella sp. IRI-160 cells were retained in each of the matrices rather than released into the surrounding medium (Table 1; FIG. 1). On the last day of the experiment, more than 80% of Shewanella sp. IRI-160 cells were embedded in each matrix, with the greatest retention (99.94%) in alginate beads. Microbes are mainly found to be associated with surfaces in nature. The attachment behaviors of bacteria may come with important benefits, including better accessibility to nutrients, as well as protection from predators and adverse environmental conditions. Studies have revealed a congregational behavior of bacteria within the genus Shewanella, that they attach to and accumulate cells surrounding insoluble iron and manganese oxides and reduce these solid-phase electron acceptors using specialized outer membrane proteins. To prepare immobilized bacteria in this study, Shewanella sp. IRI-160 was cultured in a nutrient-enriched medium and mixed with each matrix. This may have resulted in a slow release of nutrients and contributed to the matrix-bacteria association.

Alginate is a natural polymer that is characterized as low-cost, non-toxic, and highly biodegradable. In addition to its application in immobilization of other algicidal bacteria to control HABs, alginate hydrogel has been developed as edible films for food packaging, carriers for drug remote release in human bodies, and applied to neural tissue engineering. In this research, the impacts of immobilized Shewanella sp. IRI-160 on harmful dinoflagellates were investigated using bacteria embedded in alginate hydrogel beads (FIG. 2; FIG. 6; Table 2). Results of this study indicated a rapid, dose-dependent response of harmful dinoflagellates to immobilized bacteria, while no negative effects by immobilized bacteria were observed on the non-harmful control species Rhodomonas sp. (though a slight decrease of in vivo fluorescence was observed in this species treated with free-living bacteria on Day 1; FIG. 6C; Table 2). The non-negative and even slightly positive effects of immobilized Shewanella sp. IRI-160 on non-dinoflagellate species observed in this research were consistent with previous research using cell-free filtrate IRI-160AA. For instance, the cell density of Rhodomonas sp. in laboratory culture experiments and species abundance of diatom Cyclotella sp. in microcosm experiments increased when treated with algicide IRI-160AA, while this algicide was effective in controlling the growth of targeted dinoflagellates in both studies.

In this research, the growth of harmful dinoflagellates K. veneficum and P. minimum were both suppressed by the addition of immobilized and free-living Shewanella sp. IRI-160 at 10⁸ cells mL⁻¹ (FIG. 2), and as early as Day 1 (24 hours exposure; FIG. 6A-B; Table 2). Their cell densities did not recover by the end of the experiment in these treatments. These results are consistent with previous research indicating a rapid response of dinoflagellates to cell suspension or cell-free filtrate of Shewanella sp. IRI-160. For instance, it has been indicated that cell densities of K. veneficum and P. minimum declined significantly after 2 hours of exposure to cell filtrate of Shewanella sp. IRI-160. Other research reported that the cell density of K. veneficum dropped to 20 -40% of controls after 24 hours treated with cell suspension or cell-free filtrate of Shewanella sp. IRI-160.

Interestingly, although the growth of K. veneficum was inhibited by the addition of 10⁷ cells mL⁻¹ immobilized Shewanella sp. IRI-160 on Day 1, cultures in this treatment recovered by the end of the experiment, surpassing controls on Day 6 and leading to higher specific growth rates of this treatment compared to controls over the entire experiment period (FIG. 2; FIG. 6A; Table 2). A growth recovery was also observed in P. minimum treated with 10⁸ cells mL⁻¹ immobilized Shewanella sp. IRI-160 on Day 4 (FIG. 6B; Table 2). This suggests a dynamic effect of immobilized Shewanella sp. IRI-160 on dinoflagellate species, potentially involving the complex suite of algicidal compounds produced by Shewanella sp. IRI-160 in which one or more of these compounds may play a role in stimulating the growth of dinoflagellates, especially at a lower concentration (e.g. ammonium). Furthermore, results of this research imply that some portion of the dinoflagellate population may be resistant or may recover from exposure in laboratory culture experiments. However, a different response in the field may be expected. As demonstrated in natural community microcosm experiments, other phytoplankton species may outcompete dinoflagellates in communities treated with algicide IRI-160AA and/or the algicide may stimulate protistan grazers (e.g. ciliates), leading to the overall decrease of dinoflagellate abundance in natural microbial communities.

Additionally, results of this investigation demonstrated greater algicidal activity by immobilized Shewanella sp. IRI-160 against K. veneficum compared to P. minimum at the highest concentration of 10⁸ cells mL⁻¹ (FIG. 2; FIG. 6A-B; Table 2). The specific growth rates of K. veneficum treated with 10⁸ cells mL⁻¹ immobilized and free-living bacteria were negative over 6 days, while the specific growth rates of P. minimum were positive in the same treatments (FIG. 2). Furthermore, during the entire experiment period, in vivo fluorescence of K. veneficum treated with 10⁸ cells mL⁻¹ immobilized bacteria was less than 16% of that of controls, while in vivo fluorescence of the same treatments of P. minimum did not fall below 44% of controls (FIG. 6; Table 2). Immobilized Shewanella sp. IRI-160 at lower concentrations (10⁶ and 10⁷ cells mL⁻¹), however, had negative impacts on P. minimum but not K. veneficum. Overall, this indicates a varied response of dinoflagellates to immobilized Shewanella sp. IRI-160 at different densities, as well as a species-specific response of dinoflagellates to these immobilized bacteria. The higher algicidal activity of Shewanella sp. IRI-160 against K. veneficum compared to P. minimum was consistent with previous research indicating non-thecate dinoflagellates (e.g. K. veneficum, L. fissa, and Karenia brevis) were more susceptible to the algicide produced by Shewanella sp. IRI-160 compared to thecate dinoflagellates (e.g. P. minimum, Alexandrium tamarense, and Oxyrrhis marina). Species-specific responses to algicidal bacteria have also been described in other research. Cells of diatom Skeletonema costatum, for instance, were lysed within hours by the treatment of algicidal bacterium Kordia algicida, while diatom Chaetoceros didymus was not affected by the same treatment.

It was reported that the algicidal activity of bacterium Pseudomonas fluorescens HYK0210-5K09 against diatom Stephanodiscus hantzschii was lower when immobilized to alginate beads compared to the bacteria immobilized to polyester and cellulose sponge, or the free-living bacteria. It was also demonstrated that immobilization to alginate beads lowered the activity of algicidal bacterium Alcaligenes aquatilis F8 against cyanobacterium Microcystis aeruginosa. In the research presented here, however, there was only a slight difference (by 1.18 times) in specific rates between K. veneficum treated with free-living bacteria and bacteria immobilized in alginate beads at the same density, and no difference was observed between the same treatments of P. minimum (FIG. 2). This may be due to distinct algicidal mechanisms of these bacteria (e.g. Shewanella sp. IRI-160 vs. P. fluorescens HYK0210-5K09), or due to characteristics of the algicidal compounds produced by each species of bacteria. As noted, immobilization to alginate beads may physically separate algicidal bacteria from and limit their opportunities for direct contact with their target. Other research demonstrated that attachment of P. fluorescens HYK0210-5K09 to diatoms was required for cell lysis, while studies indicated direct contact was not required by Shewanella sp. IRI-160 to control dinoflagellate growth. Furthermore, the algicidal compounds produced by Shewanella sp. IRI-160 are likely to be small polar and water-soluble, so that the dispersion of these compounds may not be limited by the alginate matrix.

In this study, the bacterial cell abundance per bead increased when added to cultures of Rhodomonas sp. and K. veneficum (FIG. 3). The increase in immobilized bacterial densities in these two cultures can be partially attributed to the infiltration of beads by bacteria from non-axenic algal cultures, revealed by the cell density of immobilized bacteria in control cultures with blank alginate beads (FIG. 3 insert). However, when compared to the controls with blank beads, the higher increased bacterial density in beads with immobilized Shewanella sp. IRI-160 suggested there might be more complex processes involved, including bacteria-bacteria and/or algae-bacteria interactions. The varied growth response of immobilized bacteria in algal cultures also suggested these interactions may be species-specific, and involve different bacterial communities associated with cultures of individual algal species. These species-specific interactions were also evident in the background (free-living) bacterial densities in non-axenic algal cultures on Day 6; cultures of K. veneficum treated with immobilized and free-living Shewanella sp. IRI-160 as well as P. minimum treated with free-living Shewanella sp. IRI-160 had lower free-living bacterial densities compared to the controls, even though Shewanella sp. IRI-160 was added to these cultures on Day 0. In contrast, no difference was observed in bacterial densities of Rhodomonas sp. cultures between treatments and controls (FIG. 4). The change in the bacterial community in laboratory cultures of Pfiesteria piscicida was noted after adding free-living Shewanella sp. IRI-160. It was also demonstrated a change in prokaryotic community composition by the addition of algicide IRI-160AA to environmental samples collected during a bloom of L. fissa. Microbial interactions involving Shewanella spp. have been reported in other literature, including the antibiotic activity of Shewanella algae isolated from a marine sponge. However, it is not clear if changes in the bacterial community were in response to dinoflagellate cell death or if there were direct impacts on the bacterial community by addition of Shewanella sp. IRI-160, or both.

In aquatic environments, ammonium is thought to be the preferred inorganic nitrogen source for phytoplankton due to the low energy cost for assimilation. At high concentrations, however, it can be toxic and suppress the growth of algal species. Studies on tolerance of phytoplankton to high concentrations of ammonium suggested dinoflagellates to be the least tolerant among all algal species reviewed, including dinoflagellates, chlorophytes, diatoms, raphidophytes, and prymnesiophytes. Previous research on algicide IRI-160AA identified ammonium as one of the active algicidal compounds produced by Shewanella sp. IRI-160. A synergistic effect was observed by ammonium and n-butylamine, both present in Shewanella algicide IRI-160AA, against dinoflagellates L. fissa and P. minimum, where the combination of ammonium and n-butylamine yielded higher algicidal effects than each compound alone. This synergistic algicidal effect was not observed on Rhodomonas sp. The involvement of ammonium in the algicidal effects of bacteria was also observed in other studies. For instance, a toxic peptide (toxin P) secreted by bacterium Vibrio shiloi inhibited the photosynthesis of zooxanthellae in the presence of ammonium. The authors noted that toxin P may be able to facilitate the uptake of ammonium, which in turn disrupted the cellular pH gradient and photosynthesis.

In the study presented here, ammonium concentrations ranging from 98.2 to 565 μM were observed in treatments and controls (FIG. 7). Except for K. veneficum cultures treated with free-living and 10⁸ cells mL⁻¹ immobilized Shewanella sp. IRI-160, these concentrations were in the range for optimal growth of dinoflagellates [110±77 μM; reviewed by Collos and Harrison (2014)]. Within each species group (FIG. 5), significantly higher concentrations of ammonium were observed in K. veneficum treated with 10⁸ cells mL⁻¹ immobilized or free-living Shewanella sp. IRI-160 compared to other treatment groups. Results of experiments with P. minimum also showed that cultures with free-living Shewanella treatments had a higher ammonium concentration than controls as well as the immobilized bacteria treatments. No significant differences were observed in ammonium concentrations of controls and treatments for Rhodomonas sp. cultures. To be noted, the source of ammonium in these cultures was unknown, but may be due to remineralization of dissolved organic matter released by dead and dying dinoflagellate cells in K. veneficum and P. minimum cultures. Overall, this supports previous work suggesting that ammonium may play an important role in the algicidal effects of Shewanella sp. IRI-160 on dinoflagellates, while other essential compounds within the bacterial exudate may also be required for algicidal activity. Elucidating the role of these other compounds in the algicidal effects of Shewanella sp. IRI-160 requires future study.

### 4. Conclusion

Results of this research demonstrated good retention of Shewanella sp. IRI-160 in all matrices tested (alginate beads, agarose, sponge, and polyester cubes) at both 25 and 4° C. and indicated that association to a solid matrix may provide advantages for and protect this bacterium at warmer temperatures. Application of this technology may provide better long-term control compared to the dispersal of free-living Shewanella since dinoflagellate blooms often occur at higher temperatures. Shewanella sp. IRI-160 immobilized in alginate beads demonstrated rapid negative effects on harmful dinoflagellates K. veneficum and P. minimum, while no negative impacts were observed on non-harmful control cryptophyte Rhodomonas sp. There was no or just a slight difference in algicidal activities between immobilized and free-living Shewanella sp. IRI-160 when added at the same cell density, suggesting that diffusion of algicidal compounds produced by this bacterium is not inhibited by the matrix. In addition to impacts on dinoflagellates, the results of this research indicated potential interactions of Shewanella sp. IRI-160 or the algicidal products of this bacterium with other microbes in laboratory cultures. Ammonium, identified as a component in the algicidal filtrate, may also play a role in restructuring the bacterial community.

Overall, the results of this research revealed that immobilized Shewanella sp. IRI-160 may serve as an environmentally friendly means to control HABs and that immobilization of Shewanella sp. IRI-160 in biodegradable material such as alginate hydrogel may provide additional advantages without releasing harmful contaminants to the environment while retaining its effectiveness. Future research efforts may focus on controlled experiments evaluating field applications of immobilized Shewanella sp. IRI-160 in areas that are at risk of harmful dinoflagellate blooms.

### EXAMPLE 2. IMMOBILIZED SHEWANELLA SP. IRI-160 FILTRATE AND APPLICATION THEREOF

Further research investigated the effectiveness of preparing alginate beads with the cell-free algicide IRI-160AA alone (Shewanella sp. IRI-160 filtrate, without bacteria), as a slow-release alternative to dosing with high concentrations of the algicide.

### 1. Material and Methods

### 1.1. Algal Stock Cultures

Stock cultures of harmful dinoflagellate Karlodinium veneficum (CCMP 2936 [National Center for Marine Algae and Microbiota]) and non-harmful control species Rhodomonas sp. (CCMP 757; cryptophyte) were cultured in natural seawater at 25° C. with f/2 nutrients (—Si; Guillard and Ryther 1962) and a salinity of 20. Cultures were kept under a 12 h: 12 h light: dark cycle with a light intensity of approximately 130 μmol photons m⁻² s⁻¹, and semi-continuously in the exponential growth phase.

### 1.2. Algicide Preparation

A single colony of Shewanella sp. IRI-160 was transferred to sterile f/2 medium with 0.5% [w/v] casein amino acids (0.5% CAA medium). The culture was grown continuously at 25° C. in 10 L carboys with air being pumped in. After10 days, the culture was filtered through 0.2 μm filters and kept at 4° C. before use.

### 1.3. Beads Preparation

Two percent alginic acid (2%) was dissolved in algicide prepared above and autoclaved to sterilize. To make alginate beads, the mixture was extruded from a sterile syringe through sterile silicone tubing into a beaker of cold, sterile 0.4 M CaCl₂ solution. The solution in the beaker was mixed at low speed during extrusion. The resultant alginate beads were approximately 5 mm in diameter. The beads were washed using sterile f/2 medium (—Si and a salinity of 20; Guillard and Ryther, 1962) twice and kept in algicide at 4° C. before use. Control beads were prepared using the same process with the addition of sterile 0.5% CAA medium (with no algicide).

### 1.4 Effects of Algicide in Beads on Dinoflagellates

To evaluate the effects of algicide in beads on dinoflagellates, alginate beads with algicide were added into cultures of K. veneficum (n=3) at a final concentration of 1% [v/v]. Control beads without algicide were added at the same concentration to K. veneficum as controls (n=3). The same process was repeated for the non-harmful control species Rhodomonas sp. In vivo fluorescence was taken from the batch cultures at the beginning of the experiment and then from each treatment and control every day for 3 days.

### 1.5 Statistical Analysis

One-way ANOVA was conducted to test the significant difference of in vivo fluorescence between treatments and controls at each time point from Day 1 to Day 3 for each species.

### 2. Results

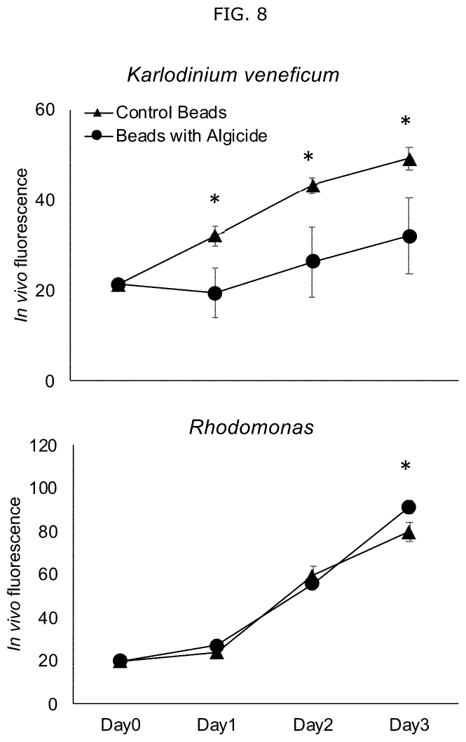

Significant differences of in vivo fluorescence of controls and treatments were observed for K. veneficum cultures on Day 1 to Day 3 (p<0.05; FIG. 8), that in vivo fluorescence of controls were 1.54 to 1.66 times higher than the ones treated with alginate beads with algicide. For Rhodomonas sp., no significant difference was observed in in vivo fluorescence of controls and treatments on Day 1 and Day 2 (p>0.05). On Day 3, Rhodomonas treatments with algicide in beads had slightly but significantly higher in vivo fluorescence than controls (p<0.05; by 1.14 times).

### 3. Conclusions

Results indicated that this approach was also effective at controlling dinoflagellate abundance in laboratory monocultures of dinoflagellate Karlodinium veneficum. No negative impacts on the control non-harmful cryptophyte Rhodomonas sp. were observed. This study suggests that alginate beads embedded with IRI-160AA, the algicidal product of Shewanella sp. IRI-160, may also be an effective and environmentally neutral approach to prevent or mitigate blooms of harmful dinoflagellates.

TABLE 1
Percent of immobilized Shewanella sp. IRI-160 (Immo.) within
each matrix (alginate beads, agarose, sponge, and polyester
cubes) and in the surrounding medium (In med.) at 25 and
4° C. at Days 0 (D 0), 3 (D 3), 6 (D 6) and 12 (D 12).

| Alginate | | Agarose | | Sponge | | Polyester | |
|---|---|---|---|---|---|---|---|
| | In | | In | | In | | In |
| Immo. | med. | Immo. | med. | Immo. | med. | Immo. | med. |
| (%) | (%) | (%) | (%) | (%) | (%) | (%) | (%) |

25° C.

| | | | | | | | | |
|---|---|---|---|---|---|---|---|---|
| D 0 | 99.99 | 0.01 | 100.00 | 0.00 | 99.99 | 0.01 | 98.20 | 1.80 |
| D 3 | 99.84 | 0.16 | 89.61 | 10.39 | 99.33 | 0.67 | 98.12 | 1.88 |
| D 6 | 99.86 | 0.14 | 90.24 | 9.76 | 98.73 | 1.27 | 97.62 | 2.38 |
| D 12 | 99.83 | 0.17 | 84.09 | 15.91 | 98.92 | 1.08 | 96.55 | 3.45 |

4° C.

| | | | | | | | | |
|---|---|---|---|---|---|---|---|---|
| D 0 | 99.99 | 0.01 | 100.00 | 0.00 | 99.99 | 0.01 | 98.20 | 1.80 |
| D 3 | 99.98 | 0.02 | 99.96 | 0.04 | 99.56 | 0.44 | 94.02 | 5.98 |
| D 6 | 99.96 | 0.04 | 97.53 | 2.47 | 99.37 | 0.63 | 96.22 | 3.78 |
| D 12 | 99.94 | 0.06 | 81.05 | 18.95 | 98.94 | 1.06 | 92.97 | 7.03 |

TABLE 2
Percentage (%) of in vivo fluorescence of treatments (harmful
dinoflagellates Karlodinium veneficum and Prorocentrum minimum, as
well as non-harmful control cryptophyte Rhodomonas sp. treated with
free-living or immobilized Shewanella sp. IRI-160 [10⁶ to 10⁸ cells mL⁻¹])
to controls (treated with blank alginate beads with no bacteria) on
Day 1 (D 1), 2 (D 2), 4 (D 4), and 6 (D 6).

| | | | | |
|---|---|---|---|---|
| Shewanella density | | | | |
| (cells mL⁻¹) | D 1 | D 2 | D 4 | D 6 |

K. veneficum

| | | | | |
|---|---|---|---|---|
| Free-living (10⁸) | *9.08 | *7.59 | *5.86 | *7.31 |
| 10⁸ | *15.90 | *7.81 | *6.71 | *9.62 |
| 10⁷ | *85.69 | 92.12 | 120.12 | *142.12 |
| 10⁶ | 100.39 | 116.51 | 109.00 | 119.15 |

P. minimum

| | | | | |
|---|---|---|---|---|
| Free-living (10⁸) | *63.57 | *65.96 | *42.44 | *61.06 |
| 10⁸ | *70.58 | *72.59 | 91.23 | *44.06 |
| 10⁷ | 98.29 | 102.94 | 94.45 | *52.43 |
| 10⁶ | 102.55 | 101.96 | *81.12 | *36.89 |

Rhodomonas sp.

| | | | | |
|---|---|---|---|---|
| Free-living (10⁸) | *86.66 | 99.95 | *133.07 | *135.56 |
| 10⁸ | 105.74 | 95.31 | *118.78 | *134.44 |
| 10⁷ | *114.65 | 99.06 | 109.32 | *118.80 |
| 10⁶ | *108.85 | 100.92 | *111.34 | *117.53 |

Asterisks “*” indicate significant differences between in vivo fluorescence of indicated group and controls (p < 0.05).

All documents, books, manuals, papers, patents, published patent applications, guides, abstracts, and/or other references cited herein are incorporated by reference in their entirety. Other embodiments of the invention will be apparent to those skilled in the art from consideration of the specification and practice of the invention disclosed herein. It is intended that the specification and examples be considered as exemplary only, with the true scope and spirit of the invention being indicated by the following claims.

1. An algicidal composition for inhibiting growth of a dinoflagellate in an environment, comprising an effective amount of Shewanella strain IRI-160 or a filtrate of a Shewanella strain IRI-160 culture, a matrix and a medium, wherein the Shewanella strain IRI-160 is immobilized to the matrix.

2. The algicidal composition of claim 1, wherein the algicidal composition comprises the Shewanella strain IRI-160 in an amount effective for maintaining a cell abundance of the dinoflagellate in the environment for at least 6 days at no more than 80% of a cell abundance of the dinoflagellate in the environment treated with a control composition, wherein the control composition comprises the matrix and the medium but not the Shewanella strain IRI-160 or the filtrate.

3. The algicidal composition of claim 1, wherein the algicidal composition comprises the Shewanella strain IRI-160 at a concentration of at least 10⁸ cells per mL.

4. The algicidal composition of claim 1, wherein the algicidal composition retains at least 80% of the Shewanella strain IRI-160 or the filtrate after storage of the algicidal composition at a temperature of 4° C. for at least 14 days.

5. The algicidal composition of claim 1, wherein the matrix comprises an agent selected from the group consisting of alginate, agarose, cellulose, polyester and a combination thereof.

6. The algicidal composition of claim 1, wherein the algicidal composition is in a form selected from the group consisting of alginate beads, agarose cubes, cellulosic sponge, polyester foam and combinations thereof.

7-12. (canceled)

13. The algicidal composition of claim 1, wherein the matrix comprises alginate.

14. The algicidal composition of claim 1, wherein the algicidal composition is in a form of alginate beads.

15. The algicidal composition of claim 1, wherein the algicidal composition is effective for inhibiting growth of the dinoflagellate in the environment after storage of the algicidal composition at a temperature of 4° C. for at least 14 days.

16. The algicidal composition of claim 1, wherein the dinoflagellate is selected from the group consisting of genus Karlodinium, Gyrodiunium, Pfiesteria, Alexandrium, Cochlodinium, Dinophysis, Karenia, Prorocentrum, Heterocapsa, and Oxyrrhis.

17. The algicidal composition of claim 1, wherein the dinoflagellate is selected from the group consisting of species Pfiesteria piscicida, Karlodinium veneficum, Alexandrium fundyense, Alexandrium tamarense, Cochlodinium polykrikoides, Cochlodinium polykrikoides, Dinophysis acuminate, Gyrodinium instriatum, Gyrodinium uncatenum, Heterocapsa triquetra, Karenia brevis, Lavenderina fissa (aka Gyrodinium instriatum and Gyrodinium uncatenum), Oxyrrhis marina and Prorocentrum minimum.

18. The algicidal composition of claim 1, wherein the medium is a casein amino acid (CAA) medium at a concentration between 1× and 10×.

19. The algicidal composition of claim 1, wherein the medium comprises natural seawater, f/2 nutrients and casein amino acids.

20. A method for preparing the algicidal composition of claim 1, comprising immobilizing the Shewanella strain IRI-160 or the filtrate in the medium to the matrix, whereby the algicidal composition is prepared.

21. (canceled)

22. The method of claim 20, wherein the matrix comprises alginate.

23. The method of claim 20, wherein the algicidal composition is in a form of alginate beads.

24. The method of claim 20, wherein the algicidal composition is effective for inhibiting growth of the dinoflagellate in the environment after storage of the algicidal composition at a temperature of 4° C. for at least 14 days.

25. The method of claim 20, wherein the dinoflagellate is selected from the group consisting of genus Karlodinium, Gyrodiunium, Pfiesteria, Alexandrium, Cochlodinium, Dinophysis, Karenia, Prorocentrum, Heterocapsa, and Oxyrrhis.

26. The method of claim 20, wherein the dinoflagellate is selected from the group consisting of species Pfiesteria piscicida, Karlodinium veneficum, Alexandrium fundyense, Alexandrium tamarense, Cochlodinium polykrikoides, Cochlodinium polykrikoides, Dinophysis acuminate, Gyrodinium instriatum, Gyrodinium uncatenum, Heterocapsa triquetra, Karenia brevis, Lavenderina fissa (aka Gyrodinium instriatum and Gyrodinium uncatenum), Oxyrrhis marina and Prorocentrum minimum.

27-28. (canceled)

29. A method for inhibiting growth of a dinoflagellate in an environment, comprising applying an effective amount of the algicidal composition of claim 1 to the dinoflagellate in the environment.

30-41. (canceled)
